(19) United States
(12) Patent Application Publication
(10) Pub. No.: US 2009/0074720 A1
(43) Pub. Date: Mar. 19, 2009

(54) METHODS FOR DECREASING IMMUNE RESPONSE AND TREATING IMMUNE CONDITIONS

(76) Inventor: Roger A. Sabbadini, Lakeside, CA (US)

Correspondence Address: Biotechnology Law Group;c/o Portfolioip, P.O. Box 52050, Minneapolis, MN, 55402, US

(21) Appl. No.: 12/098,419

(22) Filed: Apr. 5, 2008

Related U.S. Application Data
(63) Continuation-in-part of application No. 11/841,363, filed on Aug. 20, 2007.
Application No. 11/841,363 is a continuation-in-part of application No. 11/588,973, filed on Oct. 27, 2006.
Application No. 11/588,973 is a continuation-in-part of application No. 11/588,973, filed on Oct. 27, 2006.
Application No. 11/588,973 is a continuation-in-part of application No. 11/261,935, filed on Oct. 28, 2005.

(51) Int. Cl. A61K 39/395; A61P 37/00; A61K 38/43; A61P 25/00; A61K 38/21

(52) U.S. Cl. 424/85.6; 424/130.1; 424/141.1; 424/133.1; 424/94.1

(57) ABSTRACT

The present invention relates to compositions and methods for decreasing an immune response in an animal comprising administering to said animal an agent that binds a bioactive lipid and reduces the effective concentration of said bioactive lipid. Also provided are methods for treating diseases or conditions, including autoimmune disorders, which are characterized by an aberrant, excessive or undesired immune response. The methods of the invention utilize agents that bind bioactive lipids and are capable of decreasing the effective concentration of the bioactive lipid. In some embodiments, the agent is a monoclonal antibody that is reactive against sphingosine-1-phosphate (S1P) or lysophosphatidic acid (LPA).

### RELATED APPLICATIONS

This application is a continuation-in-part of commonly owned U.S. patent application Ser. Nos. 11/841,363, filed 20 Aug. 2007; 11/784,417, filed 6 Apr. 2007 (which claims priority to and the benefit of U.S. patent application Ser. Nos. 60/810,185 (filed 31 May 2006), 60/835,569 (filed 12 Aug. 2006), and 11/588,973 (filed 27 Oct. 2006); and 11/588,973, filed 27 Oct., 2006, which claims priority to: U.S. provisional patent application Ser. Nos. 60/854,971 and 60/855,003, both filed on 27 Oct. 2006; U.S. provisional patent application Ser. No. 60/835,569, filed 12 Aug. 2006; and U.S. patent application Ser. No. 11/261,935, filed 28 Oct. 2005. This application claims priority to, the benefit of, and incorporates by reference for all purposes all of the aforementioned applications, each in its entirety.

### TECHNICAL FIELD

The present invention relates to methods of decreasing an immune response using agents that bind bioactive lipid molecules and thus decrease the effective concentration of these bioactive lipid molecules. These bioactive lipids play a role in human and/or animal disease as signaling molecules. One class of bioactive signaling lipids considered in accordance with the invention is the lysolipids. Examples of signaling lysolipids are sphingosine-1-phosphate (S1P) and the various lysophosphatidic acids (LPAs). Antibodies and other agents that bind signaling lipids, and derivatives and variants thereof, thereby decreasing the effective concentration of these lipids, can be used to decrease an immune response, and in the treatment and/or prevention of diseases and conditions characterized by an excessive, aberrant or undesired immune response, through the delivery of pharmaceutical compositions that contain such antibodies, alone or in combination with other therapeutic agents and/or treatments. Autoimmune disorders, allograft rejection and graft-versus-host disease are examples of diseases and conditions which may be treated according to the methods of the present invention. Disorders characterized by inappropriate or aberrant lymphocyte infiltration are also considered to be diseases characterized by an excessive, aberrant or undesired immune response and therefore may be treated according to the methods of the present invention.

### BACKGROUND OF THE INVENTION

### I. Introduction

The following description includes information that may be useful in understanding the present invention. It is not an admission that any such information is prior art, or relevant, to the presently claimed inventions, or that any publication specifically or implicitly referenced is prior art or even particularly relevant to the presently claimed invention.

### II. Background

The present invention relates to methods of decreasing or attenuating aberrant, excessive or undesired immune responses, including autoimmune responses. These processes, separately or together, are involved in many diseases and conditions. These diseases or conditions may be systemic or may be relatively localized, for example to the red blood cells, blood vessels, connective tissues, nervous system, major organs, endocrine glands such as the thyroid or pancreas, muscles, joints or skin.

A. Diseases and Conditions Characterized by an Aberrant Excessive or Undesired Immune Response

The immune system protects the body from potentially harmful substances such as microorganisms, toxins, cancer cells, and foreign blood or cells from another person or species. These antigens are destroyed by the immune response, which includes production of antibodies and sensitized lymphocytes, which are specialized white blood cells that recognize and destroy particular antigens.

1. Autoimmune Diseases and Conditions

Autoimmune disorders develop when the immune system destroys normal body tissues, which it normally would ignore. Normally, the immune system is capable of differentiating self from non-self tissue. Some lymphocytes become sensitized against self tissue cells, but this response is usually controlled or suppressed by other lymphocytes. Autoimmune disorders occur when the normal control process is disrupted. Normally, most T cells that recognize self-antigens are eliminated in the thymus, their site of origin, and never enter general circulation. The normal T cells circulate through the lymph nodes and the blood without ever responding to self-antigens. However, it is believed that patients with autoimmune disorders bear T cells that can become activated by self-antigens. Once activated, the T cell divides to produce many effector cells which attack the activating antigen. When the antigen is a self-antigen rather than a foreign antigen, serious and potentially deadly consequences result. Autoimmune responses may also occur if normal body tissue is altered so that it is no longer recognized as self.

Autoimmune disorders may result in destruction of one or more types of body tissues, abnormal growth of an organ, or changes in organ function. The disorder may affect only one organ or tissue type or may affect multiple organs and tissues, depending on the identity of the activating antigen. Organs and tissues commonly affected by autoimmune disorders include blood components such as red blood cells, blood vessels, connective tissues, nervous system, major organs, endocrine glands such as the thyroid or pancreas, muscles, joints, and skin. A person may experience multiple autoimmune disorders at the same time.

Some nonlimiting examples of confirmed or suspected autoimmune diseases and conditions include Type 1 diabetes mellitus, psoriasis, autoimmune glomerulonephritis, autoimmune hemolytic anemia, acute disseminated encephalomyelitis, Addison's disease, alopecia universalis, ankylosing spondylitis, antiphospholipid antibody syndrome, autoimmune oophoritis, autoimmune orchitis, autoimmune polyendocrine failure, Behcet disease, Berger's disease, Buergers disease, bullous pemphigus, celiac sprue, Chagas' disease, Graves disease, Goodpastures syndrome, Guillain-Barre syndrome, Hashimato's thyroiditis, chronic active hepatitis, chronic fatigue syndrome, chronic progressive hepatitis, idiopathic thrombocytopenia purpura, Jobs syndrome, psoriatic arthritis, rheumatoid arthritis, Kawasaki's disease, multiple sclerosis, myasthenia gravis, pemphigoid, pemphigus, pemphigus erythematosus, pemphigus foliaceus, pemphigus vulgaris, polymyalgia rheumatica, pulmonary fibrosis, Reiters syndrome, Reidel's thyroiditis, rheumatic fever, sarcoidosis, Sezary syndrome; scleroderma, ulcerative colitis, autoimmune hemolytic anemia, Feltys syndrome, systemic lupus erythematosus, discoid lupus erythematosus, autoimmune polyarteritis nodosa, Caplans syndrome, Crohn's disease, dysautonomia, endometriosis, hydraadenitis suppurativa, interstitial cystitis, Lyme disease, postural orthostatic tachycardia syndrome, opsoclonus myoclonus syndrome, psoriasis, Sjogren's syndrome, CREST syndrome, viral myocarditis, Wegener's granulomatosis and Wiscott-Aldrich syndrome. Some of these disorders have been confirmed to be autoimmune disorders by the presence of autoantibodies.

a. Multiple Sclerosis (MS)

MS is an inflammatory disease of the CNS; onset occurs between the ages of 20 and 40 years and in the US, 350,000 people have MS. The course of MS is generally characterized by acute exacerbations of neurologic symptoms followed by a series of relapses and remissions. These exacerbations often result in permanent neurologic deficits. Although MS is not a fatal disease, disease progression often results in functional disability and reduced quality of life. Early symptoms of the disease may include oculomotor disturbances, tremor, ataxia, spasticity, fatigue, sensory disturbances, pain syndromes, bladder or bowel dysfunction, and psychiatric disorders. Subsequent symptoms include more prominent upper motor neuron disturbances, i.e., increased spasticity, increasing para- or quadriparesis. Vertigo, incoordination, depression, emotional lability, gait abnormalities, fatigue and pain are also commonly seen.

The precise pathogenesis of MS remains unknown; it is believed that T cells in the peripheral circulation are activated by unknown antigens and cross the blood-brain barrier into the CNS. In the CNS, the T cells stimulate production of proinflammatory cytokines that go on to cause demyelination with subsequent neurologic dysfunction. In particular, CD4+ T cells and macrophages destroy oligodendrocytes, which synthesize and maintain axonal myelin sheaths in the central nervous system (CNS).

Treatment for MS patients takes various forms, including modification of the disease course, treatment of exacerbations (also called attacks, relapses, or flare-ups) and symptom management (both primary and secondary), as well as improvement of function and safety, and emotional support. Treatment of exacerbations (which are episodes of loss of function stemming from new myelin damage) is often accomplished with high doses of corticosteroids to reduce inflammation. Some steroids, such as prednisone, are generally given orally. Others, such as methylprednisolone and dexamethasone, are given intravenously.

In addition to motor paralysis and weakness, there are many primary and secondary symptoms that affect persons with MS. These include fatigue, dizziness, nausea/vomiting, constipation, erectile dysfunction, paroxysmal itching, urinary tract infections, depression, neuropathic pain/dyesthesias, spasticity, urinary frequency, bladder dysfunction and tremors. Commonly used treatments for managing one or more of these symptoms include meclizine for dizziness and nausea/vomiting, antibiotics and antimicrobials for urinary tract infections, phenazopyridine for symptomatic relief of urinary tract infections, hydroxyzine for paroxysmal itching, papaverine, sildenafil, alprostadil, vardenafil or tadalafil for erectile dysfunction, docusate, psyllium fiber, glycerin suppositories, enemas, magnesium hydroxide, sodium phosphate or bisacodyl for constipation, duloxetine hydrochloride for depression or neuropathic pain, dantrolene for spasticity, desmopressin, oxybutynin, terazosin, prazosin, darifenacin, tamsulosin, oxybutynin, trospium chloride, imipramine, propantheline bromide, solifenacin succinate or tolterodine for urinary frequency or bladder dysfunction, phenyloin or gabapentin for dyesthesias, fluoxetine, venlafaxine, sertraline, bupropion or paroxetine for depression, amitryptyline for paresthesias, methenamine for preventing urinary tract infections, tizanidine, diazepam or baclofen for spasticity, clonazepam for tremor, pain, or spasticity, isoniazid for tremor, nortriptyline for paresthesias, modafinil or fluoxetine for fatigue, carbamazepine for trigeminal neuralgia, and imipramine for pain.

Curative therapy is the ultimate goal in MS treatment; however, this remains elusive since the underlying disease mechanism is unknown and the disease presentation is heterogeneous. The Executive Committee of the Medical Advisory Board of the National MS Society has adopted the following recommendations regarding the use of the current MS disease modifying agents, such as the immunomodulators Betaseron® (beta interferon 1b), Avonex® (beta interferon 1a), Rebif® (beta interferon 1a), Copaxone® (glatiramer acetate), and Tysabri® (natalizumab); and the immunosuppressant Novantrone® (mitoxantrone). The recommendations are that immunomodulator treatment be initiated as soon as possible following the diagnosis of MS with a relapsing course. Immunosuppressants (mitoxantrone) may be considered for selected worsening and/or relapsing patients. Therapy should be continued indefinitely except in the event of clear lack of benefits, intolerable side effects, new data or better therapy. Thus there is an unmet medical need for better treatment of patients with MS.

Emerging treatments take into account the different immunopathological mechanisms as well as strategies to protect against axonal damage or to promote remyelination.

The immunosuppressant fingolimod (FTY720 or 2-amino-2-(2-[4-octylphenyl]ethyl)-1,3-propanediol hydrochloride) has shown to exert considerable therapeutic effects in clinical trials involving patients with relapsing multiple sclerosis. Patients who received oral fingolimod once daily had a rapid reduction in disease activity, reflected in significant reductions in the relapse rate and in the number of CNS lesions. FTY720 also interferes with T-cell migration and prevents lymphocytes from leaving lymph nodes and other tissues. The sequestration of T and B lymphocytes in lymphoid tissues results in the nearly complete disappearance of lymphocytes from the blood and this process is reversible indicating that fingolimod does not kill lymphocytes. As FTY720 enters the bloodstream, it is rapidly phosphorylated. Phosphorylated FTY720 binds to all S1P receptors except S1P2. S1P1, the predominant S1P receptor expressed on lymphocytes, is a major regulator of lymphocyte migration and is required for extravascular lymphocytes to emigrate from tissues. In the brain, T cells are reactivated and induce a detrimental inflammatory reaction. FTY720 at 0.1 mg/kg p.o. or higher doses almost completely prevents paralysis in experimental autoimmune encephalomyelitis (EAE) induced by myelin basic protein in LEW rats. Therapeutic treatment with FTY720 inhibits EAE relapse induced by myelin proteolipid protein immunization in SJL mice. Webb, M et al. (2004) J. Neuroimm. 153:108:121.
Natalizumab (Tysabri®), a monoclonal antibody that blocks the α4β1 integrin-dependent adhesion of blood-borne encephalitogenic T cells and macrophages to microvessels in the CNS block T cell migration. In the peripheral lymph nodes of patients with multiple sclerosis, autoreactive CD4+ T cells are thought to encounter antigen-presenting dendritic cells and differentiate into effector cells. The encephalitogenic T cells leave the peripheral lymph nodes, gain access to the bloodstream and adhere to endothelial cells in the CNS, a step that is blocked by natalizumab.

Applicant's a murine monoclonal anti-S1P antibody, LT1002, has demonstrated modulation of lymphocyte levels in mouse. This data suggests that LT1002 could be a potentially beneficial therapeutic for auto-immune diseases and may provide a novel immunotherapeutic strategy for the treatment of MS.

2. Other Conditions Characterized by an Aberrant, Excessive or Undesired Immune Response

Methods of the present invention are also believed to be useful in treating conditions or diseases, other than autoimmune conditions, in which it is desirable to decrease or attenuate the immune response. Such conditions may be characterized by an immune response which is excessive, aberrant or undesired. Non-limiting examples include allograft rejection and graft-versus-host disease. Allografting is transplantation of an organ or tissue (e.g., kidney, heart, lung, cornea, skin, bone marrow, pancreas or other tissues or organs) into a genetically non-identical member of the same species. Thus, most human organ and tissue transplants are allografts (with the majority of the remainder being transplants from an identical twin). Allograft rejection occurs when the transplant recipient's immune system recognizes the allograft as foreign and begins to destroy it. This may eventually destroy the transplanted organ and may result in the need for a second transplant. Thus, while not necessarily unexpected, allograft rejection is an example of an immune response that is undesired.

Graft-versus-host disease (GVHD) is a complication of bone marrow transplantation and stem cell transplant. Following a bone marrow or stem cell allograft, the transplanted donor cells, e.g., T cells, may attack the patient's (the host's) body. GVHD may be chronic or acute, and may be life-threatening if uncontrolled. Thus, GVHD is an example of an undesired and/or aberrant immune response.

Lymphocyte infiltration occurs in many diseases and conditions including cancers, vascular injury, spinal cord injury, allergy and asthma. Schottenfeld and Beebe-Dimmer (2006) CA 56: 69. Zhu et al. (2002) Arteriosclerosis, Thrombosis Vasc Biol. 22: 450; (2002) Jones et al. (2002) J. Neurosci. 22: 2690; Gaga et al., (1991) J Immunol. 147:816-22; Boushey H A and J V Fahy (1995) Environ Health Perspect. 103 Suppl 6:229-233. Disorders characterized by inappropriate or aberrant lymphocyte infiltration are also considered to be diseases characterized by an excessive, aberrant or undesired immune response and therefore may be treated according to the methods of the present invention.

In certain hematological cancers, such as multiple myeloma, a malignancy of B-cells and plasma cells, treatment often involves both anti-cancer (e.g., cytotoxic) agents and immunosuppressants such as dexamethasone to reduce the aberrant immune response, (i.e., B-cell proliferation). A monoclonal antibody that binds S1P with high affinity and specificity has been shown to slow tumor progression and associated angiogenesis in several animal models of human cancer. Visentin et al., (2006) Cancer Cell 9: 225-238. The applicants believe that the anti-S1P antibody could be effective as an anti-cancer agent by virtue not only of its anti-tumorigenic activity, but also because it may be immunosuppressant. It is believed to be particularly useful for treatment of multiple myeloma and other hematological malignancies characterized by an aberrant or unwanted involvement, infiltration or proliferation of lymphocytes and their products.

B. Bioactive Signaling Lipids

Lipids and their derivatives are now recognized as important targets for medical research, not as just simple structural elements in cell membranes or as a source of energy for β-oxidation, glycolysis or other metabolic processes. In particular, certain bioactive lipids function as signaling mediators important in animal and human disease. Although most of the lipids of the plasma membrane play an exclusively structural role, a small proportion of them are involved in relaying extracellular stimuli into cells. These lipids are referred to as “bioactive lipids” or, alternatively, “bioactive signaling lipids.” “Lipid signaling” refers to any of a number of cellular signal transduction pathways that use cell membrane lipids as second messengers, as well as referring to direct interaction of a lipid signaling molecule with its own specific receptor. Lipid signaling pathways are activated by a variety of extracellular stimuli, ranging from growth factors to inflammatory cytokines, and regulate cell fate decisions such as apoptosis, differentiation and proliferation. Research into bioactive lipid signaling is an area of intense scientific investigation as more and more bioactive lipids are identified and their actions characterized.

Examples of bioactive lipids include the eicosanoids (including the cannabinoids, leukotrienes, prostaglandins, lipoxins, epoxyeicosatrienoic acids, and isoeicosanoids), non-eicosanoid cannabinoid mediators, phospholipids and their derivatives such as phosphatidic acid (PA) and phosphatidylglycerol (PG), platelet activating factor (PAF) and cardiolipins as well as lysophospholipids such as lysophosphatidyl choline (LPC) and various lysophosphatidic acids (LPA). Bioactive signaling lipids also include the sphingolipids such as sphingomyelin, ceramide, ceramide-1-phosphate, sphingosine, sphingosylphosphoryl choline, sphinganine, sphinganine-1-phosphate (Dihydro-S1P) and sphingosine-1-phosphate. Sphingolipids and their derivatives represent a group of extracellular and intracellular signaling molecules with pleiotropic effects on important cellular processes. Other examples of bioactive signaling lipids include phosphatidylserine (PS), phosphatidylinositol (PI), phosphatidylethanolamine (PEA), diacylglyceride (DG), sulfatides, gangliosides, and cerebrosides.

1. Lysolipids

Lysophospholipids (LPLs), also known as lysolipids, are low molecular weight (typically less than about 500 dalton) lipids that contain a single hydrocarbon backbone and a polar head group containing a phosphate group. Some lysolipids are bioactive signaling lipids. Two particular examples of medically important bioactive lysolipids are LPA (glycerol backbone) and S1P (sphingoid backbone). The structures of selected LPAs, S1P, and dihydro S1P are presented below.

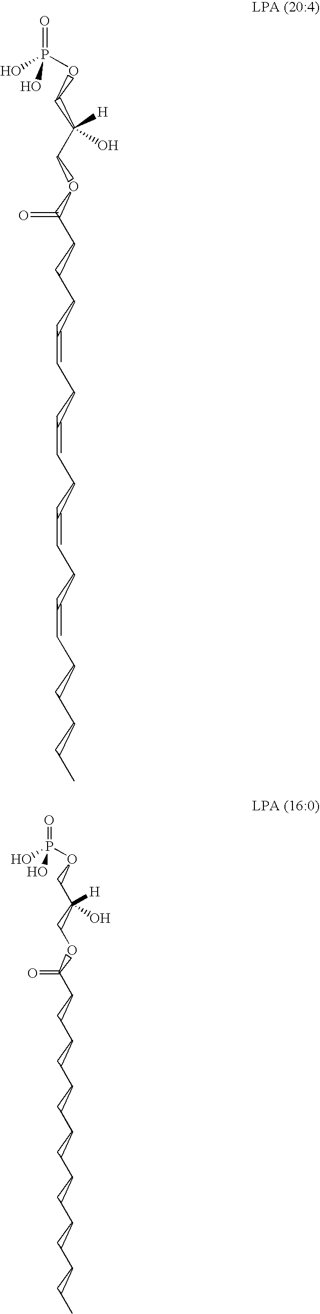

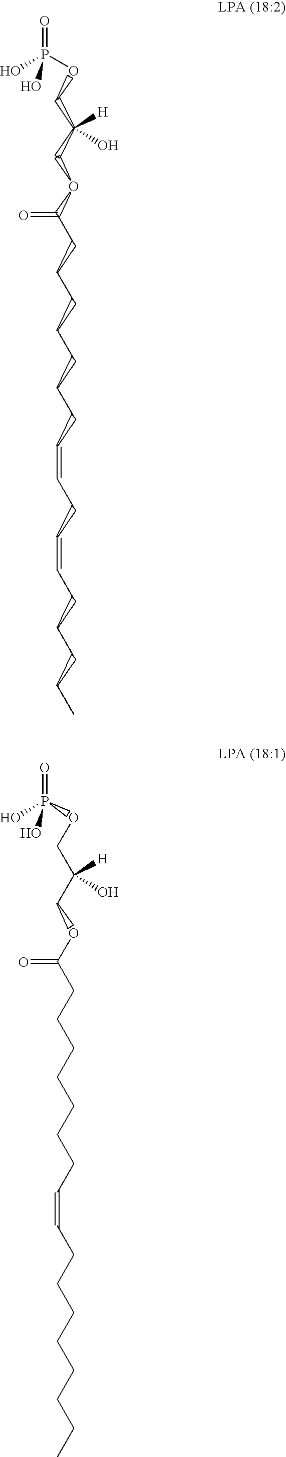

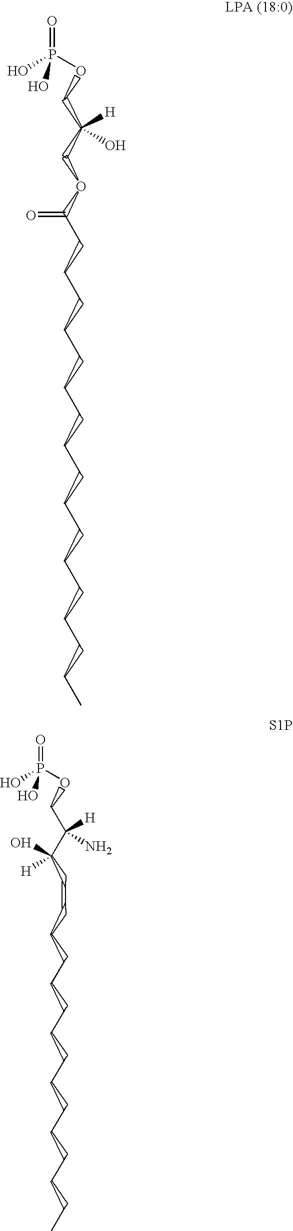

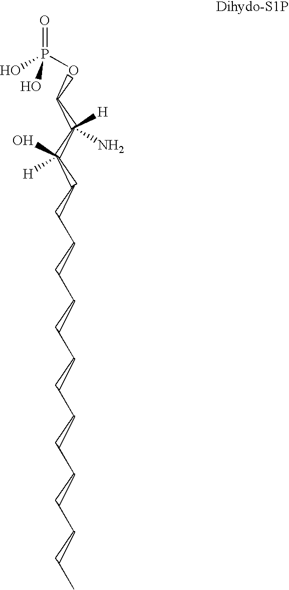

LPA is not a single molecular entity but a collection of endogenous structural variants with fatty acids of varied lengths and degrees of saturation. Fujiwara et al. (2005), J Biol Chem, 280: 35038-35050. The structural backbone of the LPAs is derived from glycerol-based phospholipids such as phosphatidylcholine (PC) or phosphatidic acid (PA). In the case of lysosphingolipids such as S1P, the fatty acid of the ceramide backbone is missing. The structural backbone of S1P, dihydro S1P (DHS1P), and sphingosylphosphorylcholine (SPC) is based on sphingosine, which is derived from sphingomyelin.

LPA and S1P regulate various cellular signaling pathways by binding to the same class of multiple transmembrane domain G protein-coupled (GPCR) receptors. Chun J, Rosen H (2006), Current Pharm Des, 12: 161-171 and Moolenaar W H (1999), Experimental Cell Research, 253: 230-238. The S1P receptors are designated as S1P1, S1P2, S1P3, S1P4 and S1P5 (formerly EDG-1, EDG-5/AGR16, EDG-3, EDG-6 and EDG-8) and the LPA receptors designated as LPA1, LPA2, LPA3 (formerly, EDG-2, EDG-4, and EDG-7). A fourth LPA receptor of this family has been identified for LPA (LPA4), and other putative receptors for these lysophospholipids have also been reported.

LPA and S1P have been shown to play a role in the immune response through modulation of immune-related cells such as T- and B-lymphocytes. These lipids promote T-cell migration to sites of immune response and regulate proliferation of T cells as well as secretion of various cytokines. Chun J and Rosen H, (2006) Curr Pharm Des. 12:161-171; Huang et al., (2002) Biophys Biochim Acta 1582:161-167; Rosen H and E J Goetzl (2005) Nat Rev Immunol (2005) 5:560-70. In particular, S1P is thought to control egress of lymphocytes into the peripheral circulation. Thus agents which bind LPA and S1P are believed to be useful in methods for decreasing an undesired, excessive or aberrant immune response, and for treating diseases and conditions, including certain hematological cancers and autoimmune disorders that are associated with an undesired, excessive or aberrant involvement of lymphocytes and or an aberrant immune response.

### a. Sphingosine-1-phosphate

S1P is a mediator of cell proliferation and protects from apoptosis through the activation of survival pathways. Maceyka et al. (2002), Biochim Biophys Acta, 1585: 192-201; Spiegel S. et al. (2003), Nat Revs Molec Cell Biol, 4: 397-407. It has been proposed that the balance between ceramide/sphingosine (CER/SPH) levels and S1P provides a rheostat mechanism that decides whether a cell is directed into the death pathway or is protected from apoptosis. The key regulatory enzyme of the rheostat mechanism is sphingosine kinase (SPHK) whose role is to convert the death-promoting bioactive signaling lipids (CER/SPH) into the growth-promoting S1P. S1P has two fates: S1P can be degraded by S1P lyase, an enzyme that cleaves S1P to phosphoethanolamine and hexadecanal, or, less common, hydrolyzed by S1P phosphatase to SPH.

S1P is abundantly generated and stored in platelets, which contain high levels of SPHK and lacks the enzymes for S1P degradation. When platelets are activated, S1P is secreted. In addition, other cell types, for example, mast cells, are also believed to be capable of secreting S1P. Once secreted, S1P is thought to be bound at high concentrations on carrier proteins such as serum albumin and lipoproteins. S1P is found in high concentrations in plasma, with concentrations in the range of 0.5-5 uM having been reported. Though primarily extracellular, intracellular actions of S1P have also been suggested (see, e.g., Spiegel S, Kolesnick R (2002), Leukemia, 16: 1596-602; Suomalainen et al (2005), Am J Pathol, 166: 773-81).

Widespread expression of the cell surface S1P receptors allows S1P to influence a diverse spectrum of cellular responses, including proliferation, adhesion, contraction, motility, morphogenesis, differentiation, and survival. This spectrum of response appears to depend upon the overlapping or distinct expression patterns of the S1P receptors within the cell and tissue systems. In addition, crosstalk between S1P and growth factor signaling pathways, including platelet-derived growth factor (PDGF), vascular endothelial growth factor (VEGF), transforming growth factor beta (TGFβ) and basic fibroblastic growth factor (bFGF), have recently been demonstrated (see, e.g., Baudhuin, et al. (2004), FASEB J, 18: 341-3). Because regulation of various cellular processes involving S1P has particular impact on neuronal signaling, vascular tone, wound healing, immune cell trafficking, reproduction, and cardiovascular function, among others, it is believed that alterations of endogenous levels of S1P within these systems can have detrimental effects, eliciting several pathophysiologic conditions, including cancer, heart failure, ocular disease and infectious and autoimmune diseases. We propose that a potentially effective strategy for treating autoimmune disorders is to reduce the biologically available extracellular levels of S1P. The applicants have developed a murine monoclonal antibody that is specific for S1P. This represents the first successfully created monoclonal antibody against a bioactive signaling sphingolipid target. The antibody acts as a molecular sponge to selectively absorb S1P from the extracellular fluid, lowering the effective concentration of S1P. It selectively binds and neutralizes S1P with picomolar affinity in biologic matrices. Visentin et al., (2006) Cancer Cell 9:225-238. Interestingly, S1P is conserved across species, unlike most proteinaceous drug targets. Human S1P is identical to murine and monkey S1P, for example.

As used herein, “sphingosine-1-phosphate” or S1P refers to sphingosine-1-phosphate [sphingene-1-phosphate; D-erythro-sphingosine-1-phosphate; sphing-4-enine-1-phosphate; (E,2S,3R)-2-amino-3-hydroxy-octadec-4-enoxy]phosphonic acid; CAS 26993-30-6] and its variants, S1P and DHS1P (dihydro sphingosine-1-phosphate [sphinganine-1-phosphate; [(2S,3R)-2-amino-3-hydroxy-octadecoxy]phosphonic acid; D-Erythro-dihydro-D-sphingosine-1-phosphate; CAS 19794-97-9] and sphingosylphosphorylcholine. Variants of S1P and LPA, as used herein, include analogs and derivatives of S1P and LPA, respectively, which function similarly, or might be expected to function similarly, to the parent molecule.

Inhibition of S1P signaling yields useful immunosuppression and amelioration of autoimmune disorders:

FTY720 (FTY; Fingolimod; 2-amino-2-(2-[4-octylphenyl]ethyl)-1,3-propanediol hydrochloride), a small molecule sphingosine analog, is a novel immunosuppressive drug that acts by altering lymphocyte trafficking, resulting in peripheral blood lymphopenia and increased lymphocyte counts in lymph nodes. FTY mediates its immune-modulating effects by binding to some of the S1P receptors expressed on lymphocytes. Bohler T et al. (2005), Transplantation, 79: 492-5.

It is believed that FTY acts by an interaction with the S1P receptors, S1P1, S1P3, S1P4 and S1P5 (but not S1P2). It is believed that initially FTY activates S1P receptors and acts as a S1P agonist. Then, FTY causes an abnormal internalization of these receptors, inactivating them by removing them from the plasma membrane. Thus while it may act initially as an agonist of S1P receptors, its long-term effects are of a functional antagonist. Massberg, S and U. von Andrian (2006) New Engl. J. Med. 355:1088-1091. The drug is administered orally and a single oral dose reduced peripheral lymphocyte counts by 30-70%. FTY reduced T-cell subset, CD4(+) cells more than CD8(+) cells. Bohler et al. (2004), Nephrol Dial Transplant, 19: 702-13. FTY treated mice showed a significant prolongation of orthotopic corneal-graft survival when administered orally. Zhang et al. (2003), Transplantation, 76: 1511-3. FTY oral treatment also significantly delayed rejection and decreased its severity in a rat-to-mouse model of corneal xenotransplantation. Sedlakova et al. (2005), Transplantation, 79, 297-303. Given the known pathogenesis of allograft rejection combined with these data suggesting that modulating the effects of the S1P signaling can improve graft survival, it is believed that agents, including antibodies that bind to, and thereby decrease the effective concentration of, bioactive lipids will also be useful in treatment of allograft rejection and other conditions characterized by an aberrant, undesired or excessive immune response.

S1P1 is involved in lymphocyte trafficking and is required for egress of lymphocytes from the thymus and secondary lymphoid organs (spleen, lymph nodes and mucosal associated lymphatic tissues such as adenoids, tonsils, appendix and Peyer's patches), which are the sites of initiation of the immune response. Lymphocytes circulate from the blood to the lymph nodes and into the lymph. Egress of lymphocytes (back to circulation) from the lymph is via the thoracic duct. Lymphocytes also recirculate via the spleen. The S1P1 inhibitor FTY causes rapid lymphopenia (reduction of lymphocytes in the blood) which is striking (10-100 fold loss in several hours) and is accompanied by a reduction of lymphocytes in lymph. An increase in lymphocytes in secondary lymphoid organs and the thymus can be seen. Thus, FTY's immune suppressive effects are believed to be due to blockage of S1P1-mediated lymphocyte egress from these organs into the circulation which would deliver the lymphocytes to the site of immune response. For review see Cyster, J., (2005) Ann. Rev. Immunol. 23:127-159. This blockage of lymphocyte egress can also be referred to as lymphocyte sequestration and is believed to account for FTY's efficacy in animal models of transplant and autoimmune disorders.

Agents and antibodies that bind to S1P and prevent ligand interaction with its complement of receptors could have a similar effect to FTY but by a different mechanism. Without being limited to a particular theory, the applicants believe that agents like anti-S1P antibodies could act by preventing S1P binding to its complement of receptors on lymphocytes and other cells involved in lymphocyte trafficking. Silencing the receptors with an anti-S1P mAb would have a similar effect to FTY's ability to down-regulate receptor presence on the surface membrane of a cell. Further, it is believed that by lowering the effective concentration of S1P, the anti-S1P mAb could act to reduce the S1P gradient between lymphatic tissue and blood. This gradient might be critical for lymphocyte egress and may act in concert with S1P activation of receptors on lymphocyte surfaces.

The marginal zone of the spleen lies between the non-lymphoid red pulp and the lymphoid white pulp of the spleen. As a result, B lymphocyte cells in the marginal zone are continuously exposed to blood (and with it, antigens). The factors that direct B cells to the marginal zone are not well understood. Treatment with FTY causes displacement of B cells from the marginal zone to lymphoid follicles, leading to the conclusion that S1P1 promotes localization of marginal zone B cells to the splenic marginal zone. Cinamon et al., (2004) Nature Immunol. 5:713-720. Thus in addition to its role in lymphocyte egress, S1P signaling also plays a role in lymphoid tissue compartmentalization.

As can be seen in Example 1 herein below, the anti-S1P mAb developed by Lpath, Inc. causes lymphopenia in mice. It can be argued that by acting as a molecular sponge to reduce the effective concentration of S1P, the antibody may be depriving the S1P receptors of their ligand and reduce the S1P gradient between lymphoid tissue and the peripheral circulation. In so doing, lymphocyte egress from lymphatics and spleen may be retarded or reduced.

Multiple sclerosis (MS) is an autoimmune disease in which an immune response directed at oligodendrocytes result in focal damage to the myelin sheaths in the central nervous system (CNS). This results in severe, generally progressive, neurological impairment and disability. A small, placebo-controlled clinical trial of FTY720 has been carried out in patients with the relapsing form of MS. FTY or placebo was given orally once per day for six months and patients who received the FTY showed rapid reduction in disease activity, as measured by a significant reduction in relapse rate. A reduction in number of gadolinium-enhanced CNS lesions measured by MRI was also demonstrated. In a switching study, patients who started on placebo showed improvement when switched to FTY. Kappos et al., (2006) N. Engl. J. Med. 355:1124-1140, and review by Massberg S and von Andrian, U. (2006) N. Engl. J. Med. 355: 1088-1091.

FTY (FTY or FTY-P) has been shown to attenuate the development of dextran sulfate sodium (DSS)-induced colitis and CD4+CD62L+ T cell transfer colitis. FTY was effective in preventing body weight loss in both models, and the disease activity index and histological colitis score were significantly lower in FTY-treated mice than in the non-treated mice. In both colitis models, FTY prevented the infiltration of CD4+ T cells into the inflamed colonic lamina propria and for that reason the authors suggest FTY as a possible clinical treatment for inflammatory bowel disease (IBD). Deguchi et al., (2006) Oncol Rep. 16:699-703.

FTY is believed to interfere with S1P signaling by binding to S1P receptors. It is believed that similar effects will be obtained using agents such as Lpath's anti-S1P mAb, which bind directly to S1P and thereby decrease the effective concentration of S1P. This is also referred to as neutralizing S1P. Examples of such agents are immune-derived moieties (e.g., antibodies and antibody fragments), small molecules, aptamers, S1P receptor fragments and the like. Thus it is believed that such agents will be effective against autoimmune diseases and other diseases characterized by an aberrant, excessive or undesired immune response.

U.S. Pat. No. 6,098,631 (Holoshitz et al.) discloses methods and compositions for treating and diagnosing autoimmune diseases using compounds that inhibit proliferation and induce apoptosis, including compounds that are inhibitors of the sphingomyelin signal transduction pathway.

### b. Lysophosphatic Acids

### LPA

LPAs have long been known as precursors of phospholipid biosynthesis in both eukaryotic and prokaryotic cells, but LPAs have emerged only recently as signaling molecules that are rapidly produced and released by activated cells, notably platelets, to influence target cells by acting on specific cell-surface receptor (see, e.g., Moolenaar et al. (2004), BioEssays, 26: 870-881 and van Leewen et al. (2003), Biochem Soc Trans, 31: 1209-1212). Besides being synthesized and processed to more complex phospholipids in the endoplasmic reticulum, LPA can be generated through the hydrolysis of pre-existing phospholipids following cell activation; for example, the sn-2 position is commonly missing a fatty acid residue due to de-acylation, leaving only the sn-3 hydroxyl esterified to a fatty acid. Moreover, a key enzyme in the production of LPA, autotaxin (lysoPLD/NPP2), may be the product of an oncogene, as many tumor types up-regulate autotoxin. Brindley (2004), J Cell Biochem, 92: 900-12. The concentrations of LPA in human plasma and serum have been reported, including determinations made using sensitive and specific LC/MS procedures' Baker et al. (2001), Anal Biochem, 292: 287-295. For example, in freshly prepared human serum allowed to sit at 25° C. for one hour, LPA concentrations have been estimated to be approximately 1.2 mM, with the LPA analogs 16:0, 18:1, 18:2, and 20:4 being the predominant species. Similarly, in freshly prepared human plasma allowed to sit at 25° C. for one hour, LPA concentrations have been estimated to be approximately 0.7 mM, with 18:1 and 18:2 LPA being the predominant species.

LPAs influence a wide range of biological responses, including induction of cell proliferation, stimulation of cell migration and neurite retraction, gap junction closure, and even slime mold chemotaxis. Goetzl et al. (2002), Scient World J, 2: 324-338. The body of knowledge about the biology of LPA continues to grow as more and more cellular systems are tested for LPA responsiveness. For example:

Wound healing: It is now known that, in addition to stimulating cell growth and proliferation, LPA promote cellular tension and cell-surface fibronectin binding, which are important events in wound repair and regeneration. Moolenaar et al. (2004), BioEssays, 26: 870-881.

Apoptosis: Recently, anti-apoptotic activity has also been ascribed to LPA, and it has recently been reported that peroxisome proliferation receptor gamma is a receptor/target for LPA. Simon et al. (2005), J Biol Chem, 280: 14656-14662.

Blood vessel maturation: Autotaxin, a secreted lysophospholipase D responsible for producing LPAs, is essential for blood vessel formation during development. van Meeteren et al. (2006), Mol Cell Biol, 26: 5015-22. In addition, unsaturated LPAs were identified as major contributors to the induction of vascular smooth muscle cell dedifferentiation. Hayashi et al. (2001), Circ Res, 89: 251-8.

Edema and vascular permeability: LPA induces plasma exudation and histamine release in mice. Hashimoto et al. (2006), J Pharmacol Sci, 100: 82-7.

Inflammation: LPA acts as inflammatory mediator in human corneal epithelial cells. Zhang et al (2006), Am J Physiol, June 7. LPA participates in corneal wound healing [Liliom K et al (1998), Am. J. Physiol, 274: C1065-C1074] and stimulates the release of ROS in lens tissue [Rao et al. (2004), Molecular Visions, 10:112-121]. LPA can also re-activate HSV-1 in rabbit cornea. Martin et al. (1999), Molec Vis, 5: 36-42.

Fibrosis and scar formation: LPA inhibits TGF-mediated stimulation of type I collagen mRNA stability via an ERK-dependent pathway in dermal fibroblasts. Sato et al. (2004), Matrix Biol, 23: 353-61. Moreover, LPA have some direct fibrogenic effects by stimulating collagen gene expression and proliferation of fibroblasts. Chen, et al. (2006) FEBS Lett. 580:4737-45.

Immune response: LPA, like S1P, has been shown to play a role in the immune response through modulation of immune-related cells. These lipids promote T-cell migration to sites of immune response and regulate proliferation of T cells as well as secretion of various cytokines. Chun J and Rosen H, (2006) Curr. Pharm Des. 12:161-171; Huang et al., (2002) Biophys. Biochim. Acta 1582:161-167; Rosen H and E J Goetzl (2005) Nat Rev Immunol. (2005) 5:560-70. A recent paper (Kanda et al., (2008) Nature Imm. 9, 415-423) further implicates LPA and autotoxin in lymphocyte trafficking. Thus agents that reduce the effective concentration of LPA, such as Lpath's anti-LPA mAb, are believed to be useful in methods for decreasing an undesired, excessive or aberrant immune response, and for treating diseases and conditions, including autoimmune disorders that are associated with an undesired, excessive or aberrant immune response.

Recently, the applicants have developed several monoclonal antibodies against LPAs. Like the anti-S1P antibody, the anti-LPA antibodies can neutralize various LPAs and mitigate their biologic and pharmacologic action. Anti-LPA antibodies are, therefore, believed to be useful in prevention and/or treatment of immune-related diseases and conditions.

### III. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

An “immune-derived moiety” refers to any polyclonal or monoclonal antibody or antibody fragment, variant, or derivative.

An “anti-S1P antibody” or an “immune-derived moiety reactive against S1P” refers to any antibody or antibody-derived molecule that binds S1P.

An “anti-LPA antibody” or an “immune-derived moiety reactive against LPA” refers to any antibody or antibody-derived molecule that binds to all or one or more of the LPAs.

A “bioactive lipid” refers to a lipid signaling molecule. In general, a bioactive lipid does not reside in a biological membrane when it exerts its signaling effects, which is to say that while such a lipid species may exist at some point in a biological membrane (for example, a cell membrane, a membrane of a cell organelle, etc.), when associated with a biological membrane it is not a bioactive lipid but is instead a “structural lipid” molecule. Bioactive lipids are distinguished from structural lipids (e.g., membrane-bound phospholipids) in that they mediate extracellular and/or intracellular signaling and thus are involved in controlling the function of many types of cells by modulating differentiation, migration, proliferation, secretion, survival, and other processes. In vivo, bioactive lipids can be found in extracellular fluids, where they can be complexed with other molecules, for example serum proteins such as albumin and lipoproteins, or in “free” form, i.e., not complexed with another molecule species. As extracellular mediators, some bioactive lipids alter cell signaling by activating membrane-bound ion channels or G-protein coupled receptors that, in turn, activate complex signaling systems that result in changes in cell function or survival. As intracellular mediators, bioactive lipids can exert their actions by directly interacting with intracellular components such as enzymes and ion channels. Representative examples of bioactive lipids include LPA and S1P.

The “effective concentration” of a bioactive lipid, such as sphingosine-1-phosphate, means the amount of said bioactive lipid that is available and active in biological processes. The effective concentration of a bioactive lipid may be reduced in several ways, including reduction of actual concentration of the lipid (through decreased production or increased degradation of the lipid, for example), reduction of concentration of freely available lipid (through complexing or binding to another molecule, for example), or reduction of the activity of the lipid (by interfering with the bioactive lipid's ability to bind one or more receptors, for example). Reducing the effective concentration of a bioactive lipid may be referred to as “neutralizing” the bioactive lipid. By way of example, an antibody that binds S1P and blocks or interferes with its biological functions (signaling, for example) may be said to neutralize S1P, because the antibody binding serves to decrease the effective concentration of available S1P, preventing it from carrying out its signaling functions.

The term “therapeutic agent” means an agent for modulating immune responses, particularly undesired, excessive or aberrant immune responses, including autoimmune responses.

The term “combination therapy” refers to a therapeutic regimen that involves the provision of at least two distinct therapies to achieve an indicated therapeutic effect. For example, a combination therapy may involve the administration of two or more chemically distinct active ingredients, for example, an anti-LPA antibody and an anti-S1P antibody. Alternatively, a combination therapy may involve the administration of an immune-derived moiety reactive against a bioactive lipid and the administration of one or more other chemotherapeutic agents or medicaments. Combination therapy may, alternatively, involve administration of an anti-lipid antibody together with the delivery of another treatment, such as radiation therapy and/or surgery. Further, a combination therapy may involve administration of an anti-lipid antibody together with one or more other biological agents (e.g., anti-VEGF, TGF, PDGF, or bFGF agent), chemotherapeutic agents and another treatment such as radiation and/or surgery. In the context of combination therapy using two or more chemically distinct active ingredients, it is understood that the active ingredients may be administered as part of the same composition or as different compositions. When administered as separate compositions, the compositions comprising the different active ingredients may be administered at the same or different times, by the same or different routes, using the same of different dosing regimens, all as the particular context requires and as determined by the attending physician. Similarly, when one or more anti-lipid antibody species, for example, an anti-LPA antibody, alone or in conjunction with one or more chemotherapeutic agents are combined with, for example, radiation and/or surgery, the drug(s) may be delivered before or after surgery or radiation treatment.

“Monotherapy” refers to a treatment regimen based on the delivery of one therapeutically effective compound, whether administered as a single dose or several doses over time.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to patentable embodiments, specifically exclude the unpatentable embodiment(s). Also, the claims appended hereto are to be interpreted both to provide the broadest reasonable scope, as well as to preserve their validity. Furthermore, the claims are to be interpreted in a way that (1) preserves their validity and (2) provides the broadest reasonable interpretation under the circumstances, if one or more of the statutory requirements for patentability are amended or if the standards change for assessing whether a particular statutory requirement for patentability is satisfied from the time this application is filed or issues as a patent to a time the validity of one or more of the appended claims is questioned.

The term “pharmaceutically acceptable salt” refers to salts which retain the biological effectiveness and properties of the agents and compounds of this invention and which are not biologically or otherwise undesirable. In many cases, the agents and compounds of this invention are capable of forming acid and/or base salts by virtue of the presence of charged groups, for example, charged amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts, see Berge et al. (1977) J. Pharm. Sci., 66, 1-19.

The terms “separated”, “purified”, “isolated”, and the like mean that one or more components of a sample contained in a sample-holding vessel are or have been physically removed from, or diluted in the presence of, one or more other sample components present in the vessel. Sample components that may be removed or diluted during a separating or purifying step include, chemical reaction products, unreacted chemicals, proteins, carbohydrates, lipids, and unbound molecules.

The term “species” is used herein in various contexts, e.g., a particular species of chemotherapeutic agent. In each context, the term refers to a population of molecules, chemically indistinguishable from each other, of the sort referred in the particular context.

“Specifically associate” and “specific association” and the like refer to a specific, non-random interaction between two molecules, which interaction depends on the presence of structural, hydrophobic/hydrophilic, and/or electrostatic features that allow appropriate chemical or molecular interactions between the molecules.

Herein, “stable” refers to an interaction between two molecules (e.g., binding of an anti-LPA or anti-S1P antibody to its target bioactive lipid) that is sufficiently strong such that the interaction of the molecules can be maintained for the desired purpose or manipulation.

A “subject” or “patient” refers to an animal in which treatment can be effected by molecules of the invention. The animal may have, be at risk for, or be believed to have or be at risk for a disease or condition that can be treated by compositions and/or methods of the present invention. Animals that can be treated in accordance with the invention include vertebrates, with mammals such as bovine, canine, equine, feline, ovine, porcine, and primate (including humans and non-human primates) animals being particularly preferred examples.

A “therapeutically effective amount” (or “effective amount”) refers to an amount of an active ingredient, e.g., an agent according to the invention, sufficient to effect treatment when administered to a subject or patient. Accordingly, what constitutes a therapeutically effective amount of a composition according to the invention may be readily determined by one of ordinary skill in the art. In the context of therapy for autoimmune or other immune-related disorders, a therapeutically effective amount is one that produces an objectively measured change in one or more parameters associated with an immune response. Nonlimiting examples of such parameters include: number of circulating T cells or lymphocytes, sequestration (e.g., accumulation) of T cells in the lymphoid organ(s), and level of lymphocyte activation.

Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of the active ingredient when administered as a monotherapy (i.e., a therapeutic regimen that employs only one chemical entity as the active ingredient).

The term “treatment” or “treating” of a disease or disorder includes preventing or protecting against the disease or disorder (that is, causing the clinical symptoms not to develop); inhibiting the disease or disorder (i.e., arresting or suppressing the development of clinical symptoms; and/or relieving the disease or disorder (i.e., causing the regression of clinical symptoms). As will be appreciated, it is not always possible to distinguish between preventing and suppressing a disease or disorder since the ultimate inductive event or events may be unknown or latent. Accordingly, the term “prophylaxis” will be understood to constitute a type of treatment that encompasses both preventing and suppressing. The term treatment thus includes prophylaxis.

The term “therapeutic regimen” means any treatment of a disease or disorder using chemotherapeutic drugs, radiation therapy, surgery, gene therapy, DNA vaccines and therapy, antisense-based therapies including siRNA therapy, anti-angiogenic therapy, immunotherapy, bone marrow transplants, aptamers and other biologics such as antibodies and antibody variants, receptor decoys and other protein-based therapeutics.

### SUMMARY OF THE INVENTION

In accordance with the present invention, methods are provided for decreasing an immune response in an animal, including a human, comprising administering to the animal an agent that binds a bioactive lipid and reduces the effective concentration of the bioactive lipid. The immune response is generally an aberrant, excessive or undesired immune response, and may be an autoimmune response.

Also provided are methods of treating diseases or conditions characterized by an aberrant, excessive or undesired immune response, comprising administering an agent that binds a bioactive lipid and reduces the effective concentration of said bioactive lipid. The disease or condition may be an autoimmune disease or condition or an undesired tissue rejection reaction. Disorders characterized by inappropriate or aberrant lymphocyte infiltration are also considered to be diseases characterized by an excessive, aberrant or undesired immune response and therefore may be treated according to the methods of the present invention.

In some embodiments of these methods, the bioactive lipid may be a sphingolipid or sphingolipid metabolite or a lysolipid or lysolipid metabolite, including S1P, LPA or a variant thereof. In some embodiments the agent that binds the bioactive lipid is an antibody, such as a monoclonal antibody, which may be a humanized monoclonal antibody. The agent may be an antibody fragment or another type of agent as described herein below.

These and other aspects and embodiments of the invention are discussed in greater detail in the sections that follow.

### BRIEF DESCRIPTION OF THE FIGURES

This document contains at least one figure executed in color. Copies of this document with color figure(s) will be provided upon request and payment of the necessary fee. A brief summary of each of the figures is provided below.

FIG. 1 is a graph that plots mean EAE score versus days post-EAE induction.

FIG. 2 contains four graphs that show the percent purity of various formulation samples that contain 11 mg/mL of the LT1009 antibody. The data plotted were obtained via SE-HPLC, and the results of the studied formulations at time zero, 0.5 month, 1 month and 2 months. On each graph, the abscissa indicates the percent-purity of each depicted data point. Starting from the bottom, the first 6 points on the ordinate in each graph are the results for pH 6.0; points 7 to 12 are the results for pH 6.5; and points 13 to 18 depict the results for pH 7.0. Likewise, the first three points from the bottom show the results for 200 ppm polysorbate-80 at pH 6.0, the next three points (4 to 6) depict the results for 500 ppm polysorbate-80 at pH 6.0, the next three points (7 to 9) depict the results for 200 ppm polysorbate-80 at pH 6.5, etc. The effect of the salt condition is depicted in groups of three. The first point of each group from the bottom represents the 148 mM NaCl condition, the next point (2) represents the 300 mM NaCl condition and the third point from the bottom represents the 450 mM NaCl condition and so on.

### DETAILED DESCRIPTION OF THE INVENTION

One way to control the amount of undesirable bioactive signaling lipids is by providing a composition that binds one or more of these lipids. The present invention describes methods for decreasing an immune response and for treating conditions associated with an aberrant, unwanted or excessive immune response. These methods comprise administering an agent that binds to a bioactive signaling lipid and decreases the effective concentration of the bioactive lipid. Antibodies and other compounds that bind to bioactive signaling lipids may be used as therapeutic sponges that reduce the effective level of lipid. When a compound is stated to be free, the compound is not in any way restricted from reaching the site or sites where it exerts its undesirable effects. Typically, a free compound is present in the cardiovascular system or lymphatics, which either is or contains the site(s) of action of the free compound, or from which a compound can freely migrate to its site(s) of action. A free compound may also be available to be acted upon by any enzyme that converts the compound into an undesirable compound.

I. Agents Useful in the Invention

A. Immune-Derived Moieties

Several antibodies have recently been approved for therapeutic use in humans by the Federal Drug Administration. Kling (1999) Mod. Drug Disc. 2:33 45. In one aspect of lipid-based therapy, antibodies that bind bioactive signaling lipids can be delivered to a patient, e.g., incorporated into pharmaceutical compositions, medical devices, and the like, for use in therapy. Such methods may work by, e.g., modulating the effective concentration of a target bioactive lipid in tissues or bodily fluids, or by removing target lipid from blood in vivo or ex vivo.

The term “immune-derived moiety”, which includes antibodies (Ab) or immunoglobulins (Ig), refers to any form of a peptide, polypeptide derived from, modeled after or encoded by, an immunoglobulin gene, or a fragment of such peptide or polypeptide that is capable of binding an antigen or epitope [see, eg, Immunobiology, 5th Edition, Janeway, Travers, Walport, Shlomchiked. (editors), Garland Publishing (2001)]. In the present invention, the antigen is a bioactive lipid molecule. Antibody molecules or immunoglobulins are large glycoprotein molecules with a molecular weight of approximately 150 kDa, usually composed of two different kinds of polypeptide chain. One polypeptide chain, termed the “heavy” chain (H) is approximately 50 kDa. The other polypeptide, termed the “light” chain (L), is approximately 25 kDa. Each immunoglobulin molecule usually consists of two heavy chains and two light chains. The two heavy chains are linked to each other by disulfide bonds, the number of which varies between the heavy chains of different immunoglobulin isotypes. Each light chain is linked to a heavy chain by one covalent disulfide bond. In any given naturally occurring antibody molecule, the two heavy chains and the two light chains are identical, harboring two identical antigen-binding sites, and are thus said to be divalent, i.e., having the capacity to bind simultaneously to two identical molecules.

The light chains of antibody molecules from any vertebrate species can be assigned to one of two clearly distinct types, kappa (κ) and lambda (λ), based on the amino acid sequences of their constant domains. The ratio of the two types of light chain varies from species to species. As a way of example, the average κ to λ ratio is 20:1 in mice, whereas in humans it is 2:1 and in cattle it is 1:20.

The heavy chains of antibody molecules from any vertebrate species can be assigned to one of five clearly distinct types, called isotypes, based on the amino acid sequences of their constant domains. Some isotypes have several subtypes. The five major classes of immunoglobulin are immunoglobulin M (IgM), immunoglobulin D (IgD), immunoglobulin G (IgG), immunoglobulin A (IgA), and immunoglobulin E (IgE). IgG is the most abundant isotype and has several subclasses (IgG1, 2, 3, and 4 in humans). The Fc fragment and hinge regions differ in antibodies of different isotypes, thus determining their functional properties. However, the overall organization of the domains is similar in all isotypes.

The term “variable region” refers to the N-terminal portion of the antibody molecule or a fragment thereof. In general, each of the four chains has a variable (V) region in its amino terminal portion, which contributes to the antigen-binding site, and a constant (C) region, which determines the isotype. The light chains are bound to the heavy chains by many noncovalent interactions and by disulfide bonds and the V regions of the heavy and light chains pair in each arm of antibody molecule to generate two identical antigen-binding sites. Some amino acid residues are believed to form an interface between the light- and heavy-chain variable domains [see Kabat et al. (1991), Sequences of Proteins of Immunological Interest, Fifth Edition, National Institute of Health, Bethesda, Md. and Clothia et al. (1985), J. Mol. Biol, vol 186: 651].

Of note, variability is not uniformly distributed throughout the variable domains of antibodies, but is concentrated in three segments called “complementarity-determining regions” (CDRs) or “hypervariable regions” both in the light-chain and the heavy-chain variable domains. The more highly conserved portions of variable domains are called the “framework region” (FR). The variable domains of native heavy and light chains each comprise four FR regions connected by three CDRs. The CDRs in each chain are held together in close proximity by the FR regions and, with the CDRs from the other chains, form the antigen-binding site of antibodies [see Kabat et al. (1991), Sequences of Proteins of Immunological Interest, Fifth Edition, National Institute of Health, Bethesda, Md.]. Collectively, the 6 CDRs contribute to the binding properties of the antibody molecule for the antigen. However, even a single variable domain (or half of an Fv, comprising only three CDRs specific for an antigen) has the ability to recognize and bind antigen [see Pluckthun (1994), in The Pharmacology of Monoclonal Antibodies, vol. 113, Rosenburg and Moore eds., Springer-Verlag, New York, pp. 269-315].

The term “constant domain” refers to the C-terminal region of an antibody heavy or light chain. Generally, the constant domains are not directly involved in the binding properties of an antibody molecule to an antigen, but exhibit various effector functions, such as participation of the antibody in antibody-dependent cellular toxicity. Here, effector functions refer to the different physiological effects of antibodies (e.g., opsonization, cell lysis, mast cell, basophil and eosinophil degranulation, and other processes) mediated by the recruitment of immune cells by the molecular interaction between the Fc domain and proteins of the immune system. The isotype of the heavy chain determines the functional properties of the antibody. Their distinctive functional properties are conferred by the carboxy-terminal portions of the heavy chains, where they are not associated with light chains.

As used herein, “antibody fragment” refers to a portion of an intact antibody that includes the antigen binding site or variable regions of an intact antibody, wherein the portion can be free of the constant heavy chain domains (e.g., CH2, CH3, and CH4) of the Fc region of the intact antibody. Alternatively, portions of the constant heavy chain domains (e.g., CH2, CH3, and CH4) can be included in the antibody fragment. Examples of antibody fragments are those that retain antigen-binding and include Fab, Fab, F(ab)2, Fd, and Fv fragments; diabodies; triabodies; single-chain antibody molecules (sc-Fv); minibodies, nanobodies, and multispecific antibodies formed from antibody fragments. By way of example, a Fab fragment also contains the constant domain of a light chain and the first constant domain (CH1) of a heavy chain.

The term “variant” refers to an amino acid sequence which differs from the native amino acid sequence of an antibody by at least one amino acid residue or modification. A “native” or “parent” or “wild-type” amino acid sequence refers to the amino acid sequence of an antibody found in nature. Variants of the antibody molecule include, but are not limited to, changes within a variable region or a constant region of a light chain and/or a heavy chain, including the hypervariable or CDR region, the Fc region, the Fab region, the CH1 domain, the CH2 domain, the CH3 domain, and the hinge region.

The term “specific” refers to the selective binding of an antibody to its target epitope. Antibody molecules can be tested for specificity of binding by comparing binding of the antibody to the desired antigen to binding of the antibody to unrelated antigen or analog antigen or antigen mixture under a given set of conditions. Preferably, an antibody according to the invention will lack significant binding to unrelated antigens, or even analogs of the target antigen. Here, the term “antigen” refers to a molecule that is recognized and bound by an antibody molecule or immune-derived moiety that binds to the antigen. The specific portion of an antigen that is bound by an antibody is termed the “epitope”. A “hapten” refers to a small molecule that can, under most circumstances, elicit an immune response (i.e., act as an antigen) only when attached to a carrier molecule, for example, a protein, polyethylene glycol (PEG), colloidal gold, silicone beads, and the like. The carrier may be one that also does not elicit an immune response by itself.

The term “antibody” is used in the broadest sense, and encompasses monoclonal, polyclonal, multispecific (e.g., bispecific, wherein each arm of the antibody is reactive with a different epitope or the same or different antigen), minibody, heteroconjugate, diabody, triabody, chimeric, and synthetic antibodies, as well as antibody fragments that specifically bind an antigen with a desired binding property and/or biological activity.

The term “monoclonal antibody” (mAb) refers to an antibody, or population of like antibodies, obtained from a population of substantially homogeneous antibodies, and is not to be construed as requiring production of the antibody by any particular method. For example, monoclonal antibodies can be made by the hybridoma method first described by Kohler and Milstein [(1975), Nature, 256: 495-497], or by recombinant DNA methods.

The term “chimeric antibody” (or “chimeric immunoglobulin”) refers to a molecule comprising a heavy and/or light chain which is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Cabilly et al. (1984), infra; Morrison et al., Proc. Natl. Acad. Sci. U.S.A. 81:6851.

The term “humanized antibody” refers to forms of antibodies that contain sequences from non-human (eg, murine) antibodies as well as human antibodies. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. Furthermore, humanized antibodies can comprise residues that are found neither in the recipient antibody nor in the imported CDR or framework sequences. These modifications are made to further refine and maximize antibody performance. Thus, in general, a humanized antibody will comprise all of at least one, and in one aspect two, variable domains, in which all or all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), or that of a human immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1; Queen et al. (1989) Proc. Natl. Acad. Sci. USA, vol 86:10029-10033).

The term “bispecific antibody” can refer to an antibody, or a monoclonal antibody, having binding properties for at least two different epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. Alternatively, bispecific antibodies can be prepared using chemical linkage. Bispecific antibodies include bispecific antibody fragments.

The term “heteroconjugate antibody” can refer to two covalently joined antibodies. Such antibodies can be prepared using known methods in synthetic protein chemistry, including using crosslinking agents. As used herein, the term “conjugate” refers to molecules formed by the covalent attachment of one or more antibody fragment(s) or binding moieties to one or more polymer molecule(s).

The term “biologically active” refers to an antibody or antibody fragment that is capable of binding the desired epitope and in some way exerting a biologic effect. Biological effects include, but are not limited to, the modulation of a growth signal, the modulation of an anti-apoptotic signal, the modulation of an apoptotic signal, the modulation of the effector function cascade, and modulation of other ligand interactions.

The term “recombinant DNA” refers to nucleic acids and gene products expressed therefrom that have been engineered, created, or modified by man. Recombinant polypeptides or proteins are polypeptides or proteins produced by recombinant DNA techniques, for example, from cells transformed by an exogenous DNA construct encoding the desired polypeptide or protein. Synthetic polypeptides or proteins are those prepared by chemical synthesis.

The term “expression cassette” refers to a nucleotide molecule capable of affecting expression of a structural gene (i.e., a protein coding sequence, such as an antibody) in a host compatible with such sequences. Expression cassettes include at least a promoter operably linked with the polypeptide-coding sequence, and, optionally, with other sequences, e.g., transcription termination signals. Additional regulatory elements necessary or helpful in effecting expression may also be used, e.g., enhancers. Thus, expression cassettes include plasmids, expression vectors, recombinant viruses, any form of recombinant naked DNA vector, and the like.

1. Antibody to S1P

Visentin et al. describe a murine monoclonal antibody that binds S1P with extremely high affinity and specificity. This antibody was shown to slow tumor progression and associated angiogenesis in several animal models of human cancer. Cancer Cell (2006) 9: 225-238.

A humanized monoclonal antibody (LT1009) has been derived from the murine anti-S1P antibody (LT1002). As compared to the murine anti-S1P antibody from which it was derived, the humanized form exhibits an S1P binding affinity in the picomolar range, as well as superior stability and in vivo efficacy. Construction, synthesis, purification, and testing of this antibody is described in U.S. patent application Ser. Nos. 60/854,971 and 11/924,890 [attorney docket nos. LPT-3010-PV and LPT-3010-UT, respectively; filed 27 Oct. 2006 and 26 Oct. 2007, respectively; each entitled “Compositions and Methods for Binding Sphingosine-1-Phosphate”], which are commonly owned with the instant invention and are hereby incorporated by reference, each in its entirety, for all purposes. It will be understood that, in general, a humanized monoclonal antibody is preferable to a murine antibody or other nonhuman-derived antibody for administration to a human subject.

2. Antibody to LPA

A monoclonal antibody against LPA has been developed. Construction, synthesis, purification, and testing of this antibody are described in U.S. patent application Ser. No. 11/755,721 (Attorney docket no. LPT-3100-UT4), which is commonly owned with the instant invention and hereby incorporated by reference in its entirety for all purposes.

3. Methods of Preparing Antibodies and Antibody Fragments and Variants

The antibodies and antibody fragments of the invention may be produced by any suitable method, for example, in vivo (in the case of polyclonal and monospecific antibodies), in cell culture (as is typically the case for monoclonal antibodies, wherein hybridoma cells expressing the desired antibody are cultured under appropriate conditions), in in vitro translation reactions, and in recombinant DNA expression systems (Johnson et al., Methods Enz. 203:88-98, 1991). Antibodies and antibody fragments and variants can be produced from a variety of animal cells, preferably from mammalian cells, with murine and human cells being particularly preferred. Antibodies that include non-naturally occurring antibody and T-cell receptor variants that retain only the desired antigen targeting capability conferred by an antigen binding site(s) of an antibody can be produced by known cell culture techniques and recombinant DNA expression systems (see, e.g., Johnson et al., Methods in Enzymol. 203:88-98, 1991; Molloy et al., Mol. Immunol. 32:73-81, 1998; Schodin et al., J. Immunol. Methods 200:69-77, 1997). Recombinant DNA expression systems are typically used in the production of antibody variants or fragments such as, e.g., bispecific antibodies and sFv molecules. Preferred recombinant DNA expression systems include those that utilize host cells and expression constructs that have been engineered to produce high levels of a particular protein. Preferred host cells and expression constructs include Escherichia coli; harboring expression constructs derived from plasmids or viruses (bacteriophage); yeast such as Sacharomyces cerevisieae or Fichia pastoras harboring episomal or chromosomally integrated expression constructs; insect cells and viruses such as Sf9 cells and baculovirus; and mammalian cells harboring episomal or chromosomally integrated (e.g., retroviral) expression constructs (for a review, see Verma et al., J. Immunol. Methods 216:165-181, 1998). Antibodies can also be produced in plants (U.S. Pat. No. 6,046,037; Ma et al., Science 268:716-719, 1995) or by phage display technology (Winter et al., Annu. Rev. Immunol. 12:433-455, 1994).

XenoMouse strains are genetically engineered mice in which the murine IgH and Igk loci have been functionally replaced by their Ig counterparts on yeast artificial YAC transgenes. These human Ig transgenes can carry the majority of the human variable repertoire and can undergo class switching from IgM to IgG isotypes. The immune system of the XenoMouse recognizes administered human antigens as foreign and produces a strong humoral response. The use of XenoMouse in conjunction with well-established hybridoma techniques results in fully human IgG mAbs with sub-nanomolar affinities for human antigens. See U.S. Pat. No. 5,770,429, entitled “Transgenic non-human animals capable of producing heterologous antibodies”; U.S. Pat. No. 6,162,963, entitled “Generation of Xenogenetic antibodies”; U.S. Pat. No. 6,150,584, entitled “Human antibodies derived from immunized XenoMice”; U.S. Pat. No. 6,114,598, entitled “Generation of xenogeneic antibodies”; and U.S. Pat. No. 6,075,181, entitled “Human antibodies derived from immunized Xenomice”; for reviews, see Green, (1999) J. Immunol. Methods 231:11-23; Wells, Chem Biol (2000) 7:R185-6; and Davis et al., (1999) Cancer Metastasis Rev; 18:421-5).

B. Receptor Fragments and Ion Channel Fragments

Soluble polypeptides derived from membrane bound, typically hydrophobic, bioactive lipid receptors that retain the receptors' ability to bind lipids may also be used to bind bioactive lipids and lipid metabolites. For example, In the case of Edg (S1P and LPA) receptors, in some instances, particular amino acid residues may be involved in the specificity of sphingolipid binding, i.e., the amino acids that determine which sphingolipid is bound by a specific receptor. Parrill et al., (2000) J. Biol. Chem. 275:39379-39384; Wang et al., (2001) J. Biol. Chem. 276:49213-49220. Such information may be used to provide soluble receptor fragments comprising receptor residues of interest, i.e., the stretches of amino acids that bind the sphingolipid. Soluble receptor fragments derived from the naturally soluble TNFalpha receptor have been prepared and at least one of these, ENBREL ((Etanercept) is in development as a therapeutic agent for arthritis. In addition, modification of such residues may permit the skilled artisan to tailor the binding specificities and/or affinity of soluble receptor fragments.

Soluble receptor fragments of particular interest include fragments of Edg-1, Edg-3, Edg-5, Edg-6 and Edg-8, all of which bind the undesirable sphingolipid sphingosine-1-phosphate (S-1-P). The Edg-1, Edg-3, Edg-5 receptors are of particular interest.

Soluble receptor fragments may be prepared in various ways including but not limited to proteolytic digestion of cells or cellular membrane preparations comprising the receptor [Bartfeld et al., (1979) Biochem Biophys Res Commun. 89:512-9; Borhani et al., (1991) J Mol. Biol. 218:685-9], recombinant DNA technologies [Marlovits et al., (1998 (J Mol. Recognit. 11:49-51; Huang et al., (1992) J Mol. Endocrinol. 8:137-44), or by in vitro synthesis of oligopeptides.

Other agents that may be used to bind bioactive lipids and lipid metabolites include fragments of ion channels that bear one or more S1P binding sites, e.g., TRP channels. Channel fragments that retain the S1P binding site(s) are useful agents for use in the methods of the instant invention.

C. Nucleic Acids

Traditionally, techniques for detecting and purifying target molecules have used polypeptides, such as antibodies, that specifically bind such targets. While nucleic acids have long been known to specifically bind other nucleic acids (e.g., ones having complementary sequences), aptamers (i.e., nucleic acids that bind non-nucleic target molecules) have been disclosed. See, e.g., Blackwell et al., Science (1990) 250:1104-1110; Blackwell et al., Science (1990) 250:1149-1152; Tuerk et al., Science (1990) 249:505-510; Joyce, (1989) Gene 82:83-87; and U.S. Pat. No. 5,840,867 entitled “Aptamer analogs specific for biomolecules”.

As applied to aptamers, the term “binding” specifically excludes the Watson-Crick-type binding interactions (i.e., A:T and G:C base-pairing) traditionally associated with the DNA double helix. The term “aptamer” thus refers to a nucleic acid or a nucleic acid derivative that specifically binds to a target molecule, wherein the target molecule is either (i) not a nucleic acid, or (ii) a nucleic acid or structural element thereof that is bound through mechanisms other than duplex- or triplex-type base pairing. Such a molecule is called a non-nucleic molecule herein.

Structures of Nucleic Acids

“Nucleic acids”, as used herein, refers to nucleic acids that are isolated from a natural source; prepared in vitro, using techniques such as PCR amplification or chemical synthesis; prepared in vivo, e.g., via recombinant DNA technology; or by any appropriate method. Nucleic acids may be of any shape (linear, circular, etc.) or topology (single-stranded, double-stranded, supercoiled, etc.). The term “nucleic acids” also includes without limitation nucleic acid derivatives such as peptide nucleic acids (PNAs) and polypeptide-nucleic acid conjugates; nucleic acids having at least one chemically modified sugar residue, backbone, internucleotide linkage, base, nucleoside, or nucleotide analog; as well as nucleic acids having chemically modified 5′ and/or 3′ ends; and nucleic acids having two or more of such modifications. Not all linkages in a nucleic acid need to be identical.

Nucleic acids that are aptamers are often, but need not be, prepared as oligonucleotides. Oligonucleotides include without limitation RNA, DNA and mixed RNA-DNA molecules having sequences of lengths that have minimum lengths of 2, 4, 6, 8, 10, 11, 12, 13, 14, 15, 17, 18, 19, 20, 21, 22, 23, 24 or 25 nucleotides, and maximum lengths of about 100, 75, 50, 40, 25, 20 or 15 or more nucleotides, irrespectively. In general, a minimum of 6 nucleotides, preferably 10 nucleotides, more preferably 14 to 20 nucleotides, is necessary to effect specific binding.

In general, the oligonucleotides may be single-stranded (ss) or double-stranded (ds) DNA or RNA, or conjugates (e.g., RNA molecules having 5′ and 3′ DNA clamps) or hybrids (e.g., RNA:DNA paired molecules), or derivatives (chemically modified forms thereof). However, single-stranded DNA is preferred, as DNA is often less labile than RNA. Similarly, chemical modifications that enhance an aptamer's specificity or stability are preferred.

Chemical Modifications of Nucleic Acids

Chemical modifications that may be incorporated into aptamers and other nucleic acids include, with neither limitation nor exclusivity, base modifications, sugar modifications, and backbone modifications.

Base modifications: The base residues in aptamers may be other than naturally occurring bases (e.g., A, G, C, T, U, 5MC, and the like). Derivatives of purines and pyrimidines are known in the art; an exemplary but not exhaustive list includes aziridinylcytosine, 4-acetylcytosine, 5-fluorouracil, 5-bromouracil, 5-carboxymethylaminomethyl-2-thiouracil, 5-carboxymethylaminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine (5MC), N6-methyladenine, 7-methylguanine, 5-methylaminomethyluracil, 5-methoxyaminomethyl-2-thiouracil, beta-D-mannosylqueosine, 5-methoxyuracil, 2-methylthio-N-6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. In addition to nucleic acids that incorporate one or more of such base derivatives, nucleic acids having nucleotide residues that are devoid of a purine or a pyrimidine base may also be included in aptamers.

Sugar modifications: The sugar residues in aptamers may be other than conventional ribose and deoxyribose residues. By way of non-limiting example, substitution at the 2′-position of the furanose residue enhances nuclease stability. An exemplary, but not exhaustive list, of modified sugar residues includes 2′ substituted sugars such as 2′-O-methyl-, 2′-O-alkyl, 2′-O-allyl, 2′-S-alkyl, 2′-S-allyl, 2′-fluoro-, 2′-halo, or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside, ethyl riboside or propylriboside.

Backbone modifications: Chemically modified backbones include, by way of non-limiting example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Chemically modified backbones that do not contain a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages, including without limitation morpholino linkages; siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; and amide backbones.

Preparation and Identification of Aptamers

In general, techniques for identifying aptamers involve incubating a preselected non-nucleic target molecule with mixtures (2 to 50 members), pools (50 to 5,000 members) or libraries (50 or more members) of different nucleic acids that are potential aptamers under conditions that allow complexes of target molecules and aptamers to form. By “different nucleic acids” it is meant that the nucleotide sequence of each potential aptamer may be different from that of any other member, that is, the sequences of the potential aptamers are random with respect to each other. Randomness can be introduced in a variety of manners such as, e.g., mutagenesis, which can be carried out in vivo by exposing cells harboring a nucleic acid with mutagenic agents, in vitro by chemical treatment of a nucleic acid, or in vitro by biochemical replication (e.g., PCR) that is deliberately allowed to proceed under conditions that reduce fidelity of replication process; randomized chemical synthesis, i.e., by synthesizing a plurality of nucleic acids having a preselected sequence that, with regards to at least one position in the sequence, is random. By “random at a position in a preselected sequence” it is meant that a position in a sequence that is normally synthesized as, e.g., as close to 100% A as possible (e.g., 5′-C-T-T-A-G-T-3′) is allowed to be randomly synthesized at that position (5′-C-T-T-N-G-T-3′, wherein N indicates a randomized position where, for example, the synthesizing reaction contains 25% each of A, T, C and G; or x % A, w % T, y % C and z % G, wherein x+w+y+z=100. In later stages of the process, the sequences are increasingly less randomized and consensus sequences may appear; in any event, it is preferred to ultimately obtain an aptamer having a unique nucleotide sequence.

Aptamers and pools of aptamers are prepared, identified, characterized and/or purified by any appropriate technique, including those utilizing in vitro synthesis, recombinant DNA techniques, PCR amplification, and the like. After their formation, target:aptamer complexes are then separated from the uncomplexed members of the nucleic acid mixture, and the nucleic acids that can be prepared from the complexes are candidate aptamers (at early stages of the technique, the aptamers generally being a population of a multiplicity of nucleotide sequences having varying degrees of specificity for the target). The resulting aptamer (mixture or pool) is then substituted for the starting aptamer (library or pool) in repeated iterations of this series of steps. When a limited number (e.g., a pool or mixture, preferably a mixture with less than 10 members, most preferably 1) of nucleic acids having satisfactory specificity is obtained, the aptamer is sequenced and characterized. Pure preparations of a given aptamer are generated by any appropriate technique (e.g., PCR amplification, in vitro chemical synthesis, and the like).

For example, Tuerk and Gold [Science (1990) 249:505-510] disclose the use of a procedure termed systematic evolution of ligands by exponential enrichment (SELEX). In this method, pools of nucleic acid molecules that are randomized at specific positions are subjected to selection for binding to a nucleic acid-binding protein (see, e.g., PCT International Publication No. WO 91/19813 and U.S. Pat. No. 5,270,163). The oligonucleotides so obtained are sequenced and otherwise characterized. Kinzler et al. used a similar technique to identify synthetic double-stranded DNA molecules that are specifically bound by DNA-binding polypeptides. Nucleic Acids Res. (1989) 17:3645-3653. Ellington et al. disclose the production of a large number of random sequence RNA molecules and the selection and identification of those that bind specifically to specific dyes such as Cibacron blue. Nature (1990) 346: 818-822.

Another technique for identifying nucleic acids that bind non-nucleic target molecules is the oligonucleotide combinatorial technique disclosed by Ecker, D. J. et al. [Nuc. Acids Res. 21, 1853 (1993)] known as synthetic unrandomization of randomized fragments (SURF), which is based on repetitive synthesis and screening of increasingly simplified sets of oligonucleotide analogue libraries, pools and mixtures [Tuerk, C. and Gold, L. Science 249, 505 (1990)]. The starting library consists of oligonucleotide analogues of defined length with one position in each pool containing a known analogue and the remaining positions containing equimolar mixtures of all other analogues. With each round of synthesis and selection, the identity of at least one position of the oligomer is determined until the sequences of optimized nucleic acid ligand aptamers are discovered.

Once a particular candidate aptamer has been identified through a SURF, SELEX or any other technique, its nucleotide sequence can be determined (as is known in the art), and its three-dimensional molecular structure can be examined by nuclear magnetic resonance (NMR). These techniques are explained in relation to the determination of the three-dimensional structure of a nucleic acid ligand that binds thrombin in Padmanabhan, K. et al., J. Biol. Chem. 24, 17651 (1993); Wang, K. Y. et al., Biochemistry 32, 1899 (1993); and Macaya, R. F. et al., Proc. Natl. Acad. Sci. USA 90, 3745 (1993). Selected aptamers may be resynthesized using one or more modified bases, sugars or backbone linkages. Aptamers consist essentially of the minimum sequence of nucleic acid needed to confer binding specificity, but may be extended on the 5′ end, the 3′ end, or both, or may be otherwise derivatized or conjugated.

D. Small Molecules

The term “small molecule” includes any chemical or other moiety, other than polypeptides and nucleic acids, that can act to affect biological processes. Small molecules can include any number of therapeutic agents presently known and used, or can be small molecules synthesized in a library of such molecules for the purpose of screening for biological function(s). Small molecules are distinguished from macromolecules by size. The small molecules of this invention usually have molecular weight less than about 5,000 daltons (Da), preferably less than about 2,500 Da, more preferably less than 1,000 Da, most preferably less than about 500 Da.

Small molecules include without limitation organic compounds, peptidomimetics and conjugates thereof. As used herein, the term “organic compound” refers to any carbon-based compound other than macromolecules such nucleic acids and polypeptides. In addition to carbon, organic compounds may contain calcium, chlorine, fluorine, copper, hydrogen, iron, potassium, nitrogen, oxygen, sulfur and other elements. An organic compound may be in an aromatic or aliphatic form. Non-limiting examples of organic compounds include acetones, alcohols, anilines, carbohydrates, monosaccharides, oligosaccharides, polysaccharides, amino acids, nucleosides, nucleotides, lipids, retinoids, steroids, proteoglycans, ketones, aldehydes, saturated, unsaturated and polyunsaturated fats, oils and waxes, alkenes, esters, ethers, thiols, sulfides, cyclic compounds, heterocyclic compounds, imidizoles and phenols. An organic compound as used herein also includes nitrated organic compounds and halogenated (e.g., chlorinated) organic compounds. Methods for preparing peptidomimetics are described below. Collections of small molecules, and small molecules identified according to the invention are characterized by techniques such as accelerator mass spectrometry (AMS; see Turteltaub et al., (2000) Curr Pharm Des 6:991-1007, and Enjalbal et al., (2000) Mass Spectrom Rev 19:139-61.

Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

E. Peptidomimetics

In general, a “polypeptide mimetic” (“peptidomimetic”) is a molecule that mimics the biological activity of a polypeptide, but that is not peptidic in chemical nature. While, in certain embodiments, a peptidomimetic is a molecule that contains no peptide bonds (that is, amide bonds between amino acids), the term peptidomimetic may include molecules that are not completely peptidic in character, such as pseudo-peptides, semi-peptides and peptoids. Examples of some peptidomimetics by the broader definition (e.g., where part of a polypeptide is replaced by a structure lacking peptide bonds) are described below. Whether completely or partially non-peptide in character, peptidomimetics according to this invention may provide a spatial arrangement of reactive chemical moieties that closely resembles the three-dimensional arrangement of active groups in a polypeptide. As a result of this similar active-site geometry, the peptidomimetic may exhibit biological effects that are similar to the biological activity of a polypeptide.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that may be obviated with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure, shape or reactivity. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide. Dean (1994), BioEssays, 16: 683-687; Cohen and Shatzmiller (1993), J Mol Graph, 11: 166-173; Wiley and Rich (1993), Med Res Rev, 13: 327-384; Moore (1994), Trends Pharmacol Sci, 15: 124-129; Hruby (1993), Biopolymers, 33: 1073-1082; Bugg et al. (1993), Sci Am, 269: 92-98, all incorporated herein by reference.

Specific examples of peptidomimetics are disclosed in U.S. Pat. No. 7,169,390 which is commonly assigned with the instant application and incorporated herein in its entirety. These examples are illustrative and not limiting in terms of the other or additional modifications.

F. Polypeptides and Polypeptide Derivatives

Examples of polypeptides and derivatives thereof are disclosed in U.S. Pat. No. 7,169,390 which is commonly assigned with the instant application and incorporated herein in its entirety. These examples are illustrative and not limiting in terms of the other or additional modifications.

II. Applications

The invention is drawn to methods for treating or preventing autoimmune diseases and conditions, using one or more therapeutic agents that alter the activity or concentration of one or more bioactive lipids, or precursors or metabolites thereof. The therapeutic methods and compositions of the invention act by changing the “effective concentration”, i.e., the absolute, relative, effective and/or available concentration and/or activities, of bioactive lipids. Lowering the effective concentration of a bioactive lipid may be said to neutralize the target lipid or its undesired effects, including downstream effects.

Without wishing to be bound by any particular theory, it is believed that bioactive signaling lipids, including S1P and/or LPA, and/or their metabolites or downstream effectors, may cause or contribute to the development of various diseases and disorders characterized by an aberrant, unwanted or excessive immune response. As such, the compositions and methods can be used to treat these immune-related diseases and disorders, particularly by decreasing the effective in vivo concentration of a particular target lipid, for example, S1P or LPA. In particular, it is believed that the compositions and methods of the invention are useful in treating autoimmune diseases, which by definition are characterized, at least in part, by an aberrant, excessive or unwanted immune response. Here, “unwanted” refers to an immune response that is undesired due to its involvement in a disease process, for example, an autoimmune response, or to an otherwise normal immune response which contributes to disease when present in excess, as in the case of transplant rejection or diseases characterized by inappropriate lymphocyte infiltration.

Examples of several classes of immune response-related diseases that may be treated in accordance with the invention are described below. It will be appreciated that many disease and conditions are characterized, at least in part, by multiple pathological processes and that the classifications provided herein are for descriptive convenience and do not limit the invention.

A. Reducing the Effective Concentration of Bioactive Lipids for the Treatment of Multiple Sclerosis

As discussed hereinabove, the sphingosine analog FTY720 has been shown to be effective in reducing relapses and CNS lesions in patients with multiple sclerosis, an autoimmune disorder. Because FTY is an S1P receptor antagonist, and therefore blocks S1P signaling, it is believed that agents that bind bioactive signaling lipids, such as lysolipids S1P and LPA, and reduce their effective concentration, will also demonstrate efficacy in treatment of MS and other autoimmune diseases and conditions. This can be demonstrated using animal models, including the acute experimental autoimmune encephalomyelitis (EAE) model, which is widely used as a standard animal model MS. In the rat EAE model, FTY provided nearly complete protection against the onset of EAE disease, and was accompanied by a reduction in infiltration of T cells into the spinal cord. Normally in EAE, myelin basic protein-specific T lymphocytes attack the myelinated tissue in the CNS. Inflammatory lesions in the CNS were also absent in FTY-treated animals, but present in control animals. Fujino et al., (2003) Pharm and Exp Therap. 305:70-77.

B. Reducing the Effective Concentration of Bioactive Lipids for the Treatment of Arthritis

Rheumatoid arthritis (RA) is an autoimmune disease that causes pain and disability due to joint inflammation and degradation. In two animal models of rheumatoid arthritis, FTY was compared to the anti-rheumatic compounds mizoribine and prednisolone in rat adjuvant-induced arthritis (AA) and collagen-induced arthritis (CIA) models. Efficacy of FTY720 at some doses was almost equal or higher as compared with mizoribine and prednisolone in both AA and CIA models. FTY, but not the other compounds, significantly decreased circulating lymphocyte levels in treated animals. FTY also demonstrated no abnormal side effects, leading the authors to conclude that it has a higher safety margin than the other two compounds, both of which demonstrated adverse effects. Matsuura, M. et al., (2000), Int. J. Immunopharmacol., 22:323-331. Because FTY is an S1P receptor antagonist, and therefore blocks S1P signaling, it is believed that agents that bind bioactive signaling lipids, such as lysolipids S1P and LPA, and reduce their effective concentration, will also demonstrate efficacy in treatment of RA and other autoimmune diseases and conditions.

C. Reducing the Effective Concentration of Bioactive Lipids for the Treatment of Diabetes

Type I diabetes is an autoimmune disorder in which the immune system damages and/or destroys the beta cells in the Islets of Langerhans of the pancreas, eliminating insulin production. Based on the efficacy of FTY720 in other autoimmune conditions and in prevention of allograft rejection, this compounds effect on development of autoimmune diabetes in nonobese diabetic (NOD) mice has been examined. Animals were given FTY orally starting from 4 weeks of age. Daily FTY doses prevented development of diabetes in almost all treated mice, whereas most untreated NOD mice became diabetic by 35 weeks of age. Withdrawal of FTY at 35 weeks of age led to development of diabetes within 2 weeks in five mice, whereas the remaining mice maintained diabetes-free conditions for up to 44 weeks of age. No side effect of the drug was seen throughout the treatment period. FTY720 also prevented cyclophosphamide-induced diabetes in NOD mice. This led the authors to conclude that FTY is a safe and effective treatment and that it may be useful for long term treatment of prediabetic individuals. Maki T. et al. (2002) Transplantation, 74:1684-6. Continuous oral FTY720 treatment in overtly diabetic NOD mice has also been shown to lead to complete reversal of diabetes. Maki, T. et al., (2005) Transplantation, 79:1051-5. Because FTY is an S1P receptor antagonist, and therefore blocks S1P signaling, it is believed that agents that bind bioactive signaling lipids, such as lysolipids S1P and LPA, and reduce their effective concentration, will also demonstrate efficacy in treatment of Type 1 diabetes and other autoimmune diseases and conditions.

D. Reducing the Effective Concentration of Bioactive Lipids for the Treatment of Scleroderma

Scleroderma is an autoimmune disease that causes scarring or thickening of the skin, and sometimes involves other areas of the body, including the lungs, heart, and/or kidneys. Scleroderma is characterized by the formation of scar tissue (fibrosis) in the skin and organs of the body, which can lead to thickening and firmness of involved areas, with consequent reduction in function. Today, about 300,000 Americans have scleroderma, according to the Scleroderma Foundation. One-third or less of those affected have widespread disease, while the remaining two-thirds primarily have skin symptoms. When the disease affects the lungs and causing scarring, breathing can become restricted because the lungs can no longer expand as they should. To measure breathing capability, doctors use a device that assesses forced vital capacity (FVC). In people with an FVC of less than 50 percent of the expected reading, the 10-year mortality rate from scleroderma-related lung disease is about 42 percent. One reason the mortality rate is so high is that no effective treatment is currently available.

As described in the examples of this application, existing evidence indicates that S1P and LPA are pro-fibrotic growth factors that can contribute to fibroblast activation, proliferation, and the resulting increased fibroblast activity associated with maladaptive scarring and remodeling. Moreover, potential roles for S1P and LPA in activity of skin and other types of fibroblasts have been demonstrated. For example, it has been shown that LPA stimulates the migration of murine skin fibroblasts [Hama, et al., (2004) J Biol Chem 279:17634-9], and human skin fibroblasts express several S1P receptor subtypes [Zhang, et al., (1999) Blood 93:2984-90]. In addition to the many direct effects of S1P on fibroblast activity, S1P also may have many potential indirect effects on fibroblast activity. For example, S1P may facilitate the action of other well-known pro-fibrotic factors, such as TGF-β and platelet derived growth factor (PDGF). TGF-β is one of the most widely studied and recognized contributors to fibrosis. Desmouliere, et al., (1993) J Cell Biol 122: 103-111. TGF-β upregulates SphK1 expression and activity leading to increased expression of tissue inhibitors of metalloproteinases 1 (TIMP-1), a protein that inhibits ECM degradation. Yamanaka, et al., (2004) J Biol Chem 279: 53994-54001. Increased expression of TIMP-1 is linked to interstitial fibrosis and diastolic dysfunction in heart failure patients. Heymans, et al., (2005) Am J Pathol 166: 15-25. Conversely, S1P stimulates expression and release of TGF-β Norata, et al., (2005) Circulation 111: 2805-2811. There is also distinct evidence of crosstalk between S1P and PDGF. S1P directly stimulates expression of PDGF. Usui, et al., (2004) J Biol Chem 279:12300-12311. In addition, the S1P1 receptor and the PDGF receptor bind one another and their association is necessary for PDGF activation of downstream signaling which contributes to proliferation and migration of various cell types. Long, et al., (2004) Prostaglandins Other Lipid Mediat 80: 74-80; Baudhuin et al., (2004) Faseb J 18: 341-343. As such, the effects of TGF-β and PDGF on fibrosis may be due in part to crosstalk with the S1P signaling pathway. As such, the compositions and methods of the invention can be used to treat scleroderma, particularly by decreasing the effective in vivo concentration of a particular target lipid, for example, S1P and/or LPA.

Systemic scleroderma is thought to be exacerbated by stimulatory autoantibodies against PDGF receptors [Baroni, et al., (2006) N Engl J Med. 354:2667-76], and PDGF receptors are up-regulated in scleroderma fibroblasts in response to TGF-β. Yamakage, et al., (1992) J Exp Med. 175:1227-34. Because of the substantial cross-talk among the S1P, PDGF and TGF-β signaling systems, blocking S1P bioactivity with an anti-S1P agent (e.g., an anti-S1P mAb) could indirectly mitigate the pro-sclerotic effects of PDGF and TGF-β. Moreover, treatment with such an anti-S1P agent could benefit scleroderma patients by mitigating the direct effects of S1P, including fibrosis, on skin and other forms of fibroblasts that contribute to disease progression. Thus it is believed that agents that bind bioactive signaling lipids, such as lysolipids S1P and LPA, and reduce their effective concentration, will also demonstrate efficacy in treatment of scleroderma and other autoimmune diseases and conditions, particularly those with a fibrotic component. This gives these agents a distinct advantage over therapeutic agents that modulate either fibrosis or an immune response alone. “Inflammatory scarring” is a name given to a combination of inflammation and fibrosis, originally in the context of chronic renal disease. For discussion see Peters et al., (2004), Kidney Intl. 66: 1434-1443. It is believed that agents that decrease the effective concentration of bioactive signaling lipids, will be particularly effective in conditions characterized by both a scarring and an autoimmune and/or inflammatory component.

E. Reducing the Effective Concentration of Bioactive Lipids for the Prevention and Treatment of Allograft Rejection

In animal models of corneal transplants, FTY720-treated mice showed a significant prolongation of orthotopic corneal-graft survival when administered orally. Zhang et al. (2003), Transplantation, vol 76: 1511-3. FTY oral treatment also significantly delayed rejection and decreased its severity in a rat-to-mouse model of corneal xenotransplantation. Sedlakova et al. (2005), Transplantation, vol 79, 297-303. Given the known pathogenesis of allograft rejection combined with these data suggesting that modulating the effects of the S1P signaling can improve graft survival, it is believed that agents that bind to, and thereby decrease the effective concentration of, bioactive lipids will also be useful in treatment of allograft rejection, graft-versus-host disease and other conditions characterized by an aberrant, undesired or excessive immune response.

FTY720 has been shown to prevent graft rejection and facilitate long-term graft acceptance in animal models (rat, dog) of heart, small bowel, kidney and liver allografts. In a human clinical trial of FTY in stable renal transplant patients, FTY was well tolerated and caused the expected reversible lymphopenia. Budde, K. et al., (2002) J. Am. Soc. Nephrol. 13:1073-1083. In an initial Phase 2a clinical trial to evaluate the efficacy and safety of FTY in de novo renal transplantation, in combination with mycophenolate mofetil (MMF), FTY was found to be as effective as MMF in combination with cyclosporine for the prevention of acute rejection after renal transplantation, and was well tolerated. Tedesco-Silva H., et al., (2005) Transplantation, 79: 1553-60.

Because FTY is an S1P receptor antagonist, and therefore blocks S1P signaling, it is believed that agents that bind bioactive signaling lipids, such as lysolipids S1P and LPA, and reduce their effective concentration, will also demonstrate efficacy in treatment of allograft rejection, graft-versus-host disease and other conditions characterized, at least in part, by an aberrant, excessive or unwanted immune response.

F. Reducing the Effective Concentration of Bioactive Lipids for the Prevention and Treatment of Glomerulonephritis

Immune diseases of the glomerulus, such as glomerulonephritis, are among the major causes of end-stage renal disease. These diseases share a progressive course characterized by fibrosis and inflammation of the tubulointerstitial compartment. “Inflammatory scarring” is a name given to a combination of inflammation and fibrosis, originally in the context of chronic renal disease. For discussion, see Peters, et al., (2004), Kidney Intl. 66: 1434-1443. It is believed that agents that decrease the effective concentration of bioactive signaling lipids, will be particularly effective in conditions characterized by both a scarring and an immune and/or inflammatory component.

In a rat model of glomerulonephritis, FTY720 treatment reduced circulating lymphocyte counts as well as renal lymphocyte infiltration. The course of disease progression was slowed significantly. Peters, et al., supra. Because FTY is an S1P receptor antagonist, and therefore blocks S1P signaling, it is believed that agents that bind bioactive signaling lipids, such as lysolipids S1P and LPA, and reduce their effective concentration, will also demonstrate efficacy in treatment of glomerulonephritis, other immune-based kidney diseases and other conditions characterized, at least in part, by an aberrant, excessive or unwanted immune response.

III. Methods of Administration

The treatment for diseases and conditions such as the examples given above can be administered by various routes employing different formulations and devices. Suitable pharmaceutically acceptable diluents, carriers, and excipients are well known in the art.

One skilled in the art will appreciate that the amounts to be administered for any particular treatment protocol can readily be determined. Suitable amounts might be expected to fall within the range of 10 μg/dose to 10 g/dose, preferably within 10 mg/dose to 1 g/dose.

Drug substances may be administered by techniques known in the art, including but not limited to systemic, subcutaneous, intradermal, mucosal, including by inhalation, and topical administration. The mucosa refers to the epithelial tissue that lines the internal cavities of the body. For example, the mucosa comprises the alimentary canal, including the mouth, esophagus, stomach, intestines, and anus; the respiratory tract, including the nasal passages, trachea, bronchi, and lungs; and the genitalia. For the purpose of this specification, the mucosa will also include the external surface of the eye, i.e. the cornea and conjunctiva. Local administration (as opposed to systemic administration) may be advantageous because this approach can limit potential systemic side effects, but still allow therapeutic effect.

Pharmaceutical compositions used in the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations used in the present invention may be prepared according to conventional techniques well known in the pharmaceutical industry. Such techniques include the step of bringing into association the active ingredients with the pharmaceutical carrier(s) or excipient(s). Preferred carriers include those that are pharmaceutically acceptable, particularly when the composition is intended for therapeutic use in humans. For non-human therapeutic applications (e.g., in the treatment of companion animals, livestock, fish, or poultry), veterinarily acceptable carriers may be employed. In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment the pharmaceutical compositions may be formulated and used as foams. Pharmaceutical foams include formulations such as, but not limited to, emulsions, microemulsions, creams, jellies and liposomes.

While basically similar in nature these formulations vary in the components and the consistency of the final product. The know-how on the preparation of such compositions and formulations is generally known to those skilled in the pharmaceutical and formulation arts and may be applied to the formulation of the compositions of the present invention.

Various excipients might also be added to the formulated antibody to improve performance of the therapy, make the therapy more convenient or to clearly ensure that the formulated antibody is used only for its intended, approved purpose. Examples of excipients include chemicals to control pH, antimicrobial agents, preservatives to prevent loss of antibody potency, dyes, e.g., to identify the formulation for particular route of administration only, solubilizing agents to increase the concentration of antibody in the formulation, penetration enhancers and the use of agents to adjust isotonicity and/or viscosity. Inhibitors of, e.g., proteases, could be added to prolong the half life of the antibody.

The antibody might also be chemically modified to yield a pro-drug that is administered in one of the formulations or devices previously described above. The active form of the antibody is then released by action of an endogenous enzyme. Possible ocular enzymes to be considered in this application are the various cytochrome p450s, aldehyde reductases, ketone reductases, esterases or N-acetyl-β-glucosamidases. Other chemical modifications to the antibody could increase its molecular weight, and as a result, increase the residence time of the antibody in the eye. An example of such a chemical modification is pegylation [Harris and Chess (2003), Nat Rev Drug Discov; 2: 214-21], a process that can be general or specific for a functional group such as disulfide [Shaunak et al. (2006), Nat Chem Biol; 2:312-3] or a thiol [Doherty et al. (2005), Bioconjug Chem; 16: 1291-8].

### EXAMPLES

The invention will be further described by reference to the following detailed examples. These Examples are in no way to be considered to limit the scope of the invention

### Example 1

### Effect of Agents that Decrease the Effective Concentration of Bioactive Lipids on Lymphopenia

As is summarized in Tables 1 and 2, a 28-day toxicology study with murine monoclonal antibody LT1002 (SPHINGOMAB) was performed at doses of 0, 30, 75 and 200 mg/kg. As is shown in data tables 1-7 below, there was a dose-related decline of lymphocytes at all dose levels and of basophils at the highest dose. This decline was reflected in an increase in % neutrophils, % monocytes and % reticulocytes and a parallel decrease in % lymphocytes. This decrease in circulating neutrophils parallels the effect seen with FTY720, a small molecule sphingosine analog, which is a novel immunosuppressive drug that acts by altering lymphocyte trafficking, resulting in peripheral blood lymphopenia.

TABLE 1
28-Day General Toxicology Study Design
28-Day General Toxicology Study

| | |
|---|---|
| Test Article | LT1002 |
| Species | C57Bl mouse |
| Number of Animals | 10/sex/group |
| Dose | 0, 30, 75 & 200 mg/kg |
| Route of Administration | iv Bolus by tail vein injection* |
| Duration | 28 consecutive daily administrations |
| GLP | Yes |
| Study Endpoints | Clinical observations, body & organ |
| | weights, food consumption, necropsy |
| | of 48 tissues, bone marrow smears, |
| | clinical chemistries, hematology, |
| | coagulation panels & dose formulation |
| | analyses |

*Dosing ip, instead of dosing iv, initiated on day 14 because of tail vein damage post multiple iv injections of test article

TABLE 2
Summary of Findings

| | | | Significance |
|---|---|---|---|
| Parameter | Dose Group | Effect | (p-value) |
| WBC | 30 | Reduced | 0.007 |
| | 75 | Reduced | 0.043 |
| | 200 | Reduced | 0.013 |
| HGB | 30 | Reduced | 0.037 |
| MCV | 200 | Increased | 0.040 |
| Lymph | 30 | Reduced | 0.001 |
| | 75 | Reduced | 0.003 |
| | 200 | Reduced | 0.002 |
| BASO | 30 | Reduced | 0.047 |
| NEUT (%) | 30 | Increased | 0.004 |
| | 75 | Increased | 0.001 |
| | 200 | Increased | 0.000 |
| LYMPH (%) | 30 | Reduced | 0.001 |
| | 75 | Reduced | 0.000 |
| | 200 | Reduced | 0.000 |
| MONO (%) | 30 | Increased | 0.010 |
| | 75 | Increased | 0.000 |
| | 200 | Increased | 0.000 |
| RETIC (%) | 75 | Increased | 0.042 |
| GLOB | 30 | Increased | 0.027 |
| | 75 | Increased | 0.007 |
| | 200 | Increased | 0.000 |
| A/G Ratio | 30 | Reduced | 0.012 |
| | 75 | Reduced | 0.007 |
| | 200 | Reduced | 0.000 |
| Ca | 75 | Increased | 0.045 |
| | 200 | Increased | 0.000 |
| Spleen (% of body | 200 | Reduced | 0.020 |
| weight) | | | |

### Example 2

### 28-Day Toxicology Study in Mice with Anti-S1P Monoclonal Antibody-Effect on Spleen

A 28-day study of LT1002 in mice performed by LAB Preclinical (Study 1005-2615), in which forty organs and the site of injection (tail), were evaluated for gross pathology in all Control and High Dose Level (Group 4; 200 mg/kg/day) animals. LT1002 is the murine version of LT1009, Lpath's anti-S1P monoclonal antibody.

Organs evaluated included adrenals, aorta (thoracic), brain (cerebral cortex, midbrain, cerebellum and medulla), cecum, colon, epididymides, esophagus, eyes, femur with marrow, gallbladder, heart, kidneys, liver (2 lobes), lungs with bronchi, lymph nodes (mandibular and mesenteric), mammary glands (inguinal), optic nerves, ovaries, pancreas, pituitary, prostate, rectum, salivary glands (mandibular), sciatic nerve, seminal vesicles, skeletal muscle (thigh), ski/subcutis (inguinal), small intestines (duodenum, ileum and jejunum), spinal cord (cervical, lumbar and thoracic) spleen, sternum with marrow, stomach, testes, thymus, thyroids with parathyroids, tongue, trachea, urinary bladder, uterus (horns, body and cervix) and vagina.

The following preliminary histopathological changes were noted by the LAB pathologist for Group 4 (200 mg/kg/day) animals. “Mild to moderate decreased size of the follicular marginal zone of the splenic white pulp was noted in 6/10 male and 5/10 female mice from Group 4. Although this finding did not suggest splenic lymphoid toxicity, it could not be excluded as a LT1002-related change. Decreased size of the splenic follicular marginal zone was characterized by variable narrowing of the lymphoid mantle (i.e., marginal zone), cuffing the lymphoid follicles of the white pulp.

Mild to marked increased extramedullary hematopoiesis of the splenic red pulp in 3/10 males and 5/10 females from Group 4 and mild increased extramedullary hematopoiesis of the splenic red pulp in one male and one female from Group 3 (75 mg/kg/day) (the only spleens examined in Group 3 mice) were considered potentially LT1002-related, but of no toxicological significance.”

Histopathological examination of the spleen of all mice from Groups 2 (30 mg/kg/day) and Group 3 (75 mg/kg/day), and a full histopathologic assessment of all pre-terminal mice are underway. LAB reports no other findings from the histopathologic evaluation of tissues from the Group 4 (200 mg/kg/day) animals. As per protocol, no tissues from animals in other dose groups besides the 200 mg/kg/day and saline treated animals were studied except for tissue in animals exhibiting macroscopic abnormalities (i.e. local irritation at the site of injection).

FTY720—a small molecule super agonist/functional antagonist of at least one, or more, of the GPCR receptors for S1P—is in late clinical development for the treatment of relapsing, remitting multiple sclerosis. FTY720 is thought to act in animals and man by altering lymphocyte trafficking/homing patterns. FTY also provides protection in animal models of human cancer. The long-term effects of FTY720 include systemic lymphopenia and decreased T-cell responses after 2 weeks oral administration of 1 mg/kg/day to normal female C57BL or C3H mice: lymphocytes in peripheral blood, peripheral lymph nodes, mesenteric lymph nodes, Peyer's patches, and spleen were all decreased. The long-term effects of FTY720 in mice also include a reduction in spleen weights by 65% after 2 weeks oral administration of 1 mg/kg/day to normal female C57BL or C3H mice.

In the present study, decreased spleen weights observed in LAB study 1005-2615 in animals treated with 200 mg/kg/day LT1002 is consistent with the same finding reported with daily administration of FTY720. Because FTY720 and LT1002 affect the same set of cell receptors, albeit by different mechanisms, and because the two compounds possess overlapping pharmacologic profiles, the reduction in spleen weights and spleen morphology in the present study with LT1002 was not unexpected.

### Example 3

### Effect of Agents that Decrease the Effective Concentration of Bioactive Lipids on Lymphocyte Trafficking

The S1P signaling inhibitor FTY720 is believed to act in an immunosuppressive manner by altering lymphocyte trafficking/homing patterns and acceleration of lymphocyte homing. Chiba et al., (1998) J. Immunol. 160: 5037. The effect of anti-S1P antibody on lymphocyte trafficking is also examined, essentially as in published methods. Schwab et al., (2005) Science 309: 1735-1739.

Mice are treated with murine S1P monoclonal antibody or an isotype matched control monoclonal antibody. Treatments consist of intravenous injection of antibody diluted into 200-300 μL of normal saline. Animals are sacrificed at varying times after antibody administration. Lymphocyte counts are performed in lymph nodes, spleen, thymus, blood and lymph. Antibody inhibition of S1P causes a decrease in circulating lymphocytes (i.e., lymphopenia) and a corresponding increase in lymphocytes in lymphoid organs similar to that seen after FTY treatment is expected.

### Example 4

### Efficacy of Agents that Decrease the Effective Concentration of Bioactive Lipids in an Immune Challenge Study

⁵¹Chromium-Release CTL Assays

Primary ex vivo cytotoxic lymphocyte (CTL) assays are performed using ⁵¹Cr-labeled MC-57 cells incubated in the presence or absence of the immunodominant peptide) as targets, as described in Murali-Krishna, K., et al., (1998) Immunity 8 (2): 177-87. Results are determined by applying the following equation and multiplying by 100%:

$$\frac{( Experimental lysis - spontaneous lysis )}{( Maximal lysis - spontaneous lysis )} .$$

Intracellular Cytokine Staining (ICCS)

Splenocytes (4×10⁶) are incubated for 12 hr in 250 μL of RPMI-1640 containing 10% FBS and Golgi Stop (Pharmingen, San Diego, Calif.) in the presence of 2 μg/mL of the immunodominant H-2^{b} restricted CD8+ T cell epitope peptide. Negative controls are incubated without peptide. Following stimulation, cells are stained for CD8 and intracellular IFN-γ as specified by the manufacturer (Pharmingen). Following staining, cells are analyzed by flow cytometry using a FACScan or FACSCalibur and the data analyzed for expression of CD8 and IFN-γ using CellQuest™ software (Becton Dickinson Immunocytometry Systems, San Jose, Calif.). Percent of peptide-specific activation of CD8+ T cells is calculated by dividing the number of CD8+ T cells expressing IFN-γ by the total number of CD8+ T cells. As a positive control for the induction of T cells to produce IFN-γ, an equivalent number of splenocytes from naïve control animals are incubated for 6 hr in the presence of 20 ng/mL phorbol-12-myristate-13-acetate (PMA, Calbiochem, La Jolla, Calif.) and 3 μM ionomycin (Calbiochem) prior to staining.

### Example 5

### Efficacy of Agents that Decrease the Effective Concentration of Bioactive Lipids in a Murine Experimental Allergic Encephalomyelitis (EAE) Model of Multiple Sclerosis

EAE is an experimental autoimmune disease of the central nervous system (CNS) (Zamvil et al, (1990) Ann. Rev. Immunol., 8:579 and is a disease model for the human autoimmune condition, multiple sclerosis (MS) [Alvord et al, Experimental Allergic Model for Multiple Sclerosis, NY 511 (1984)]. It is readily induced in mammalian species [for example, SJL/J mice are a susceptible strain of mice (H-2^{s})] by immunizations of myelin basic protein purified from the CNS (e.g., an emulsion of guinea pig or bovine spinal column) or an encephalitogenic proteolipid (PLP) or a peptide fragment of myelin oligodendrocyte glycoprotein (MOG₃₅₋₅₅). Experimental allergic encephalomyelitis (EAE) in mice, induced by injection of white matter, is a useful model of CNS inflammation, and has been used in the study of multiple sclerosis (Spahn et al. 1999; also see over 40 papers on various mouse models of EAE by Howard Weiner).

Animals with EAE develop an acute paralytic disease and an acute cellular infiltrate is identifiable within the CNS. Thus in addition to serving as a standard model for MS, this model has also been used to determine T-cell infiltration into the CNS. T-lymphocytes are rarely found in the normal CNS, but during MS, HIV induced encephalomyelitis or other CNS inflammatory conditions these cells are present. Symptoms observed include muscle weakness, paralysis, and lack of coordination.

Experimental protocol—the following experiments were conducted in the laboratory of Dr. Howard Weiner in collaboration with Lpath, Inc.

A peptide fragment of myelin oligodendrocyte glycoprotein (MOG₃₅₋₅₅; MEVGWYRSPFSRVVHLYRNGK) is used to cause experimental EAE in mice. Mendel, I. et al., A myelin oligodendrocyte glycoprotein peptide induces typical chronic experimental autoimmune encephalomyelitis in H-2b mice: fine specificity and T cell receptor V beta expression of encephalitogenic T cells, Eur J Immunol. 1995 July; 25 (7):1951-9. EAE was induced in mice by immunization with a peptide corresponding to amino acids 35-55 of myelin oligodendrocyte glycoprotein (MOG) peptide (MOG₃₅₋₅₅) to induce chronic experimental autoimmune encephalomyelitis (EAE). Following immunization with MOG₃₅₋₅₅, mice develop an acute episode of EAE that is followed by a gradually developing progressive disease thought to model several features of progressive multiple sclerosis. Mice were treated with antibody to S1P and treated mice were followed to study the effects of S1P-specific antibody on the clinical manifestations of this acute model of EAE. At the end of the experiment, treated mice were also used to analyze the effect of anti-S1P administration on the immune response.

In a first set of experiments, using 10 mice per group with an average weight of 20 gr, EAE was induced in C57BL/6J mice with 150 ug/mouse MOG₃₅₋₅₅ in CFA along with 150 pg/mouse pertussis toxin on days 0 and 2. Anti-S1P antibody LT1002 (Sphingomab™) was administered i.v. at a dosage of 75 mg/Kg on day −1, followed by i.p. dosing at 25 mg/Kg every 2 days. Treated mice were followed for 25 days to study the effects of S1P-specific antibodies on the clinical manifestations of this acute model of EAE. Clinical manifestations of the disease include flaccid tail, and limb paralysis which may be scored to give an objective evaluation of clinical symptoms. The standard evaluation of disease severity in the EAE model measures clinical behavior on a 0-6 scale: 0) normal; 1) flaccid tail; 2) abnormal gait, hind leg weakness; 3) partial paralysis, severe ataxia; 4) minimal hind leg movement after painful stimulus; 5) no hind leg movement; 6) moribund state with little or no movement. Means were compared between groups to determine the effect of treatment on clinical scores and body weight gain. Statistical significance of clinical scores and weight were resolved using biostatistical analysis.

At day 17, two representative mice were sacrificed to study the cellular immune response (proliferation and cytokines) and to analyze CNS pathology. Spinal cords were dissected and sectioned. Sections were stained with hematoxylin and eosin (H&E) to reveal inflammatory cells, with Luxol to visualize myelin, or with silver stain to visualize axons. Total levels of CD4+FoxP3+T_{reg} cells were evaluated by FACS analysis of blood samples taken from the tail on day 17. At day 25, treated mice were analyzed to see the effect of anti-S1P administration on the immune response.

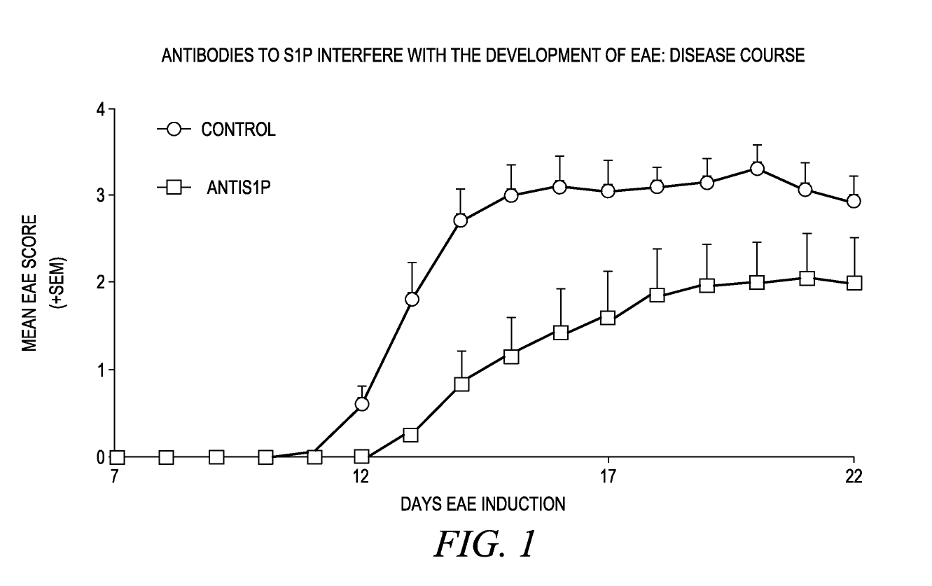

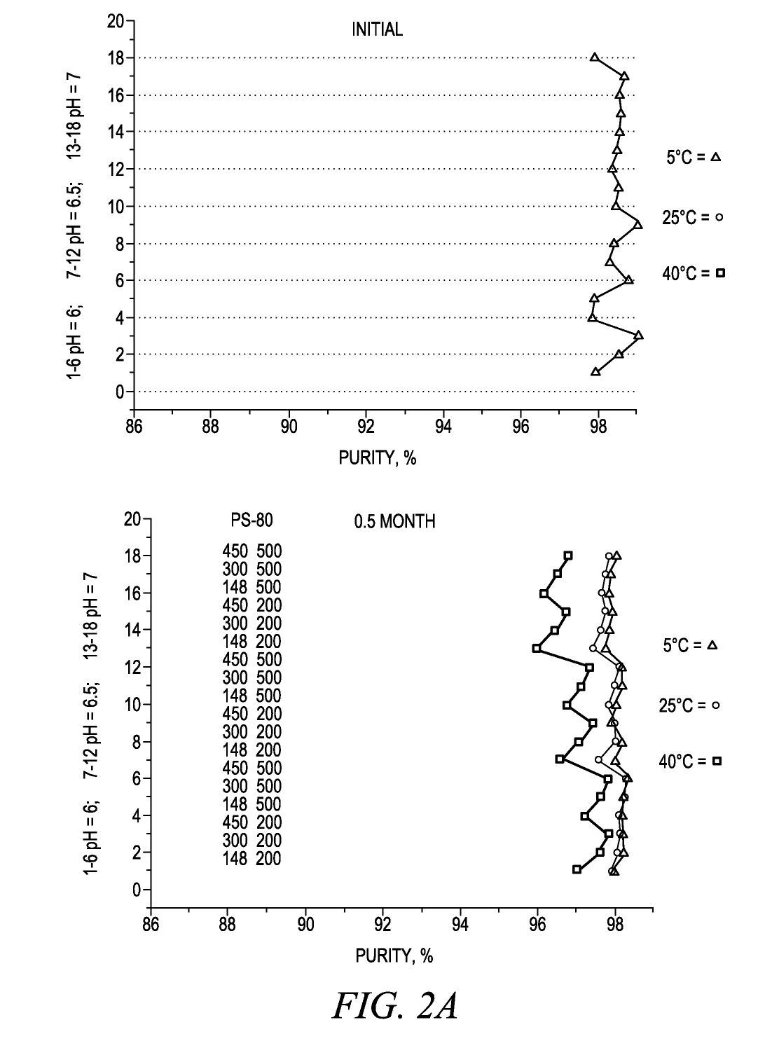

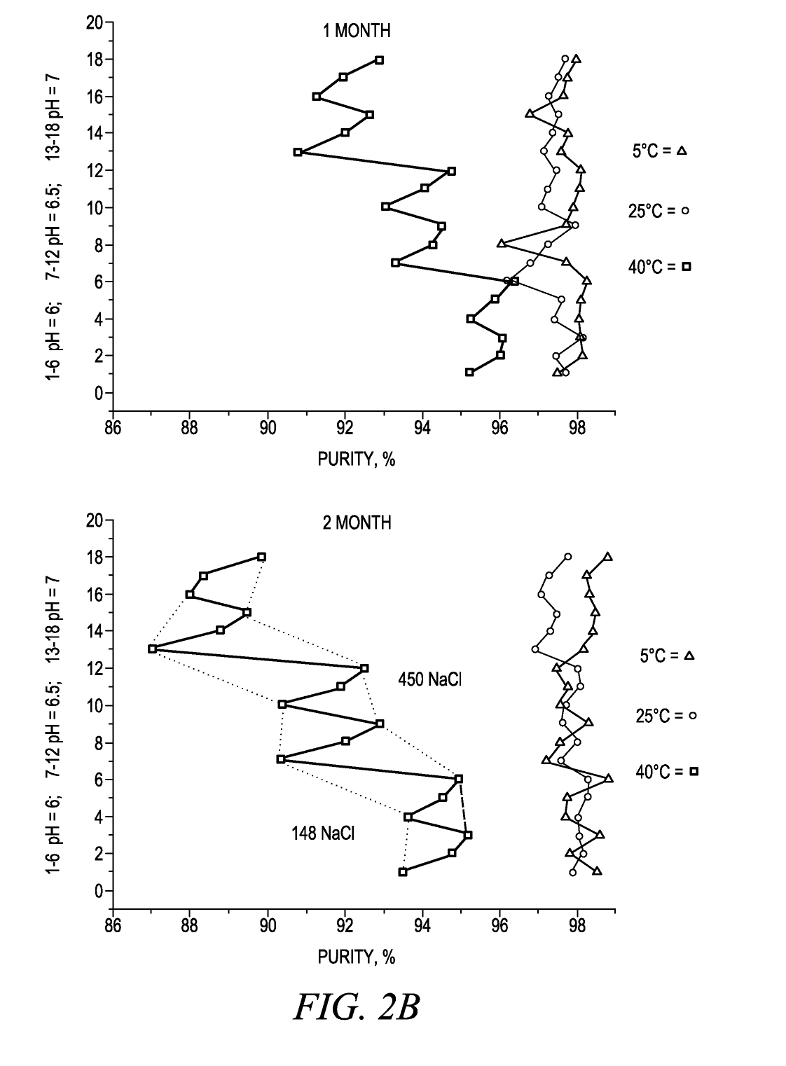

Administration of anti-S1P antibody (LT1002; Sphingomab™) was found to lead to a significant inhibition of EAE development (p<0.001). This can be seen in as a decrease in EAE symptoms, scored as described above, which is shown in FIG. 1. After antibody treatment, the EAE scores level off at approximately 2 (abnormal gait, hind leg weakness) on the scale of disease severity, whereas the untreated control animals had an EAE severity score of approximately 3 (partial paralysis, severe ataxia). This clinical effect is confirmed histologically, with a quantifiable decrease in demyelination (less than 1% demyelination in antibody-treated animals vs. 9% demyelination in untreated controls with EAE, approximately a 92% decrease after treatment), decrease in axonal loss (less than 2% axonal loss in antibody-treated mice vs. 18% axonal loss in untreated control animals with EAE, approximately a 91% decrease after treatment) and decrease of about 85% in number of inflammatory cells/mm² in spinal cords of antibody-treated animals compared to untreated controls with EAE.

In addition, anti-S1P administration led to a decrease in the number of circulating lymphocytes and spleen CD4+T cells). Circulating lymphocytes totaled approximately 10% in antibody-treated animals, and about 13.5% in control animals with EAE. This decrease was significant and represents approximately a 26% decrease in circulating lymphocytes after antibody treatment. Spleen CD4+ T cells totaled approximately 7.6% of lymphocytes in spleen in antibody-treated animals, as compared to approx. 11.7% of lymphocytes in control animals. This decrease was also significant and represents about a 35% decrease after antibody treatment.

A class of specialized T cells that inhibit the proliferation and activation of effector T cells are known as suppressor or regulatory T cells (T_{regs}). A major class of T_{regs} is CD4⁺CD25⁺ T cells which play a role in the maintenance of self tolerance. Depletion of CD4+CD25+T_{reg} in mice results in the onset of systemic autoimmune diseases, and under such conditions, murine EAE increased in severity; thus it is believed that impairment of T_{reg} cells may contribute to disease. Kumar et al., (2006) J. Neuroimm. 178:184; Sakaguchi et al. (1985) and Stephens et al., (2005), both as cited in Kumar et al. Kumar et al. also found a reduction or loss of suppressive activity of T_{reg} cells from MS patients.

CD4⁺CD25⁺T_{regs} express the transcription factor Foxp3 gene at relatively high levels. CD25+ and Fox P3+ together are specific markers for T_{reg} cells. Anti-S1P administration to mice with EAE was found to be associated with a significant increase in CD4⁺CD25⁺Foxp3⁺T_{reg} in spleen and a similar but statistically nonsignificant increase in circulating CD4⁺CD25⁺Foxp3⁺T_{reg} cells. These results indicate an increase in T_{reg} cells after antibody treatment.

Injection of synthetic MOG peptide (amino acids 35-55 of myelin oligodendrocyte glycoprotein, referred to as MOG₃₅₋₅₅) causes mice to develop significant specific T cell responses in addition to neurological symptoms. Spahn et al., (1999) Eur. J. Immunol. 29:4060-4071. Anti-S1P administration was associated with a significant decrease in this proliferative recall response to MOG₃₅₋₅₅. Mice were primed by injections of MOG peptide. The spleens were removed and homogenized, and splenocytes were pulsed with different amounts of MOG peptide. Subsequently, cells were pulsed with radiolabel and incubated before cells were harvested and label incorporation was assessed by scintillation counting. T cell priming to MOG was found to be reduced after S1P antibody treatment. As measured in cpm, the recall proliferative response was decreased by approximately 36%, 38% and 22% after anti-S1P antibody treatment (at 4 ug/ml, 20 ug/ml and 100 ug/ml of MOG₃₅₋₅₅, respectively). This blockade of lymphocyte proliferation by the S1P antibody suggests that the antibody is able to block the autoimmune response to MOG in these mice.

Splenocytes from anti-S1P-treated mice secreted lower levels of IL-17 and IFNγ and higher levels of IL-10 upon stimulation with MOG₃₅₋₅₅. Using methods essentially as in Spahn et al. (1999) Eur. J. Immunol. 29:4060-4071, the protective cytokine IL-10 was increased by antibody treatment, compared to control splenocytes from untreated animals, while the deleterious cytokines are decreased.

Thus it has been shown that treatment with antibody to S1P decreases symptoms of EAE, a well-accepted animal model of MS. Paralysis and weakness are decreased, and this is supported by histological findings showing neurodegeneration, demyelination and inflammation in the CNS. Anti-S1P administration also led to a significant decrease in the number of circulating lymphocytes and spleen CD4+ T cells, indicating the effect of this antibody on lymphocyte trafficking. Anti-S1P administration was also associated with a significant increase in regulatory T cells. Thus the anti-S1P antibody acts through several mechanisms, all of which are believed to be useful in reduction of disease indicators in this model of MS.

Anti-S1P antibody (LT1002; Sphingomab™) is also expected to be efficacious in the SJL/PLP (relapsing-remitting) interventional model of MS. EAE is induced in SJL/J mice (10 mice/group) by immunization with a peptide corresponding to the 139-151 aa region of the proteolipid protein (PLP139-151). See Webb et al. (2004) J. Neuroimmunol. 153:108-121. Following immunization with PLP139-151, SJL/J mice develop a disease thought to model several features of multiple sclerosis. Antibody treatment is given after the neurological symptoms appear. The same doses of the anti-S1P antibody (LT1002) will be used as in the experiment above: 75 mg/kg on day 17 i.v. (peak of disease) followed by a dose of 25 mg/kg every two days.

### Example 6

### Efficacy of Agents that Decrease the Effective Concentration of Bioactive Lipids in a Collagen-Induced Arthritis (CIA) Model of Rheumatoid Arthritis

Collagen-induced arthritis (CIA) is an animal model for the human autoimmune disease rheumatoid arthritis (RA). Trenthorn et al, (1977) J. Exp. Med., 146:857. This disease can be induced in many species by the administration of heterologous type II collagen [Courtenay et al, (1980) Nature, 283:665; Cathcart et al, (1986) Lab. Invest., 54:26], and this is an accepted model for study of the disease.

Collagen-induced arthritis (CIA) in the mouse is induced by immunization of susceptible mice strains with native type II collagen. Macroscopically evident arthritis occurs between days 28-35 after immunization and persists for several months until the joints ankylose. CIA shares several histopathologic features with RA including mononuclear cell infiltration and synovial cell hyperplasia with bone and cartilage destruction. In both RA and CIA, disease susceptibility is restricted by MHC class II alleles and autoreactive T cells are prominent in the joint. Because of these similarities, CIA is a widely used experimental model for RA. Typically, CIA is induced on day 1 in 6-7 week-old male mice by intradermal tail base injection of bovine or chicken collagen II (CII) supplemented with 2.0 mg/ml M. tuberculosis emulsified in complete Freund's adjuvant (CFA). On day 21, mice receive an intradermal tail base injection of CII in incomplete Freund's adjuvant. Clinical severity of disease is evaluated every 4 days. Each paw is scored for inflammation on a scale of 0-4: 0, normal; 1, erythema and mild swelling confined to ankle, or tarsals, or individual digits; 2, moderate erythema and swelling of tarsals and ankle; 3, severe erythema and mild swelling of ankle, tarsals and digits; 4, severe erythema and severe swelling of ankle, tarsals and digits. Total daily scores for each mouse is obtained by adding scores from all four paws. On day 60 mice are euthanized and forepaws are weighed. For histology, paws are fixed with 10% formalin, decalcified in Decal (Fisher), embedded in paraffin, and 5 μm sections are stained with hematoxylin/eosin.

Collagen-induced arthritis and adjuvant-induced arthritis (AA) are widely used animal models for the evaluation of new anti-arthritic drugs. The disease development of these models is also accepted for T-cell-dependent counterparts of human rheumatoid arthritis. For example, anti-CD4 antibody suppresses the disease development of AA and CIA, indicating that CD4+-positive T cells play a major part in the induction of AA and CIA.

Another accepted mouse model for arthritis is the TNF transgene model. Transgenic mice expressing a modified human TNF-α transgene spontaneously develop a chronic polyarthritis providing further evidence for the direct involvement of TNF in the pathogenesis of human RA. Mice carrying a human TNF transgene with a modified 3′ region from a human globin gene show deregulated human TNF expression resulting in low level expression of TNF in the joints and a variety of other organs. In contrast, mice carrying a wild type human TNF transgene showed appropriately regulated TNF expression. Mice with deregulated TNF expression developed a chronic symmetric polyarthritis with histologic features similar to human RA. This process does not require a specific genetic background in the target mice. Other well accepted animal models for RA are reviewed in Kannan, K. et al., (2005), Pathophysiology 12:167-181.

The efficacy of anti-S1P monoclonal antibody is evaluated in the CIA animal model of rheumatoid arthritis. Age 6-8 week old male DBA1/J mice are purchased from Jackson Laboratory (Bar Harbor, Me.). They are fed with high-fat content mouse chow (Purina mouse chow 5015) and given deionized water, ad libitum.

Animals are randomized to treatment groups (10 mice/group) based on body weight. Animals receive vehicle (saline), anti-S1P monoclonal antibody or positive control (dexamethasone) beginning on Day 21. Dosing is p.o. except for dexamethasone which is given s.c. Three dose groups (low, medium and high) of antibody are used. All test articles are tested for the presence of endotoxin because LPS contamination can have a stimulating effect on the disease progression and may interfere with the evaluation of drug efficacy. Severity scores/paw volumes are measured three times per week beginning on Day 21 and continuing until Day 42.

To induce arthritis, chick type II collagen in Complete Freund's adjuvant (CFA) (mixed 1:1) is administered to all animals by intradermal route at the tail on Day 1 and boosted with type II collagen in Incomplete Freund's adjuvant (IFA) on Day 21. Beginning on Day 21 and continuing through Day 42, clinical severity score and paw volumes are measured on two hind paws three times per week. Paw thickness is measured using a calibrated caliper. Body weights are monitored weekly. General cage side observations are monitored at least once per day. At necropsy, both hind paws are collected and preserved in 10% buffered formalin. Hind paws collected at the necropsy are subject to microradiography using a Faxitron machine.

Paw/ankle are decalcified in formic acid until radiotranslucent. Four micron sections are prepared and stained with Safarin O and tartrate resistant acid phosphatase. The histology sections are qualitatively assessed for their extent of inflammation, articular cartilage damage, bone resorption and destruction, and synovial tissue changes.

### Example 7

### Efficacy of Agents that Decrease the Effective Concentration of Bioactive Lipids in a Mouse Model of Type 1 Diabetes

It has been shown that treatment of nonobese diabetic (NOD) mice with FTY720 prevents the onset of diabetes. Continuous oral FTY720 treatment in overtly diabetic NOD mice can also result in reversal of diabetes. See Maki et al., supra. It is believed that agents, such as anti-S1P monoclonal antibody, that decrease the effective concentration of bioactive lipids will have a similar effect on diabetes. This will be tested in standard NOD mouse models using standard methods.

### Example 8

### Efficacy of Agents that Decrease the Effective Concentration of Bioactive Lipids in a Murine Scleroderma Model

Scleroderma, a debilitating acquired connective tissue disease, is characterized by fibrosis, particularly of the skin and lungs. A murine sclerodermatous graft-vs.-host disease (Scl GVHD) model for scleroderma has been developed for the study of basic immunologic mechanisms that drive fibrosing diseases and GVHD itself. This model reproduces important features of scleroderma including skin thickening, lung fibrosis, and up-regulation of cutaneous collagen mRNA, which is preceded by monocyte infiltration and the up-regulation of cutaneous TGF-1 mRNA. McCormick, L. L. (1999) J. Immunol. 163: 5693-5699. Briefly, recipient mice are lethally irradiated and then injected with allogeneic donor spleen and bone marrow cell suspension. Sclerodermatous thickening of skin is detectable by day 21 post-BMT by image analysis of routine histopathological sections. Other animal models for scleroderma are discussed in a review by Varga: Lakos G, Takagawa S, Varga J. (2004) Methods Mol. Med. 102:377-93.

Anti-S1P antibody or other agents that bind and reduce the effective concentration of bioactive lipid are administered by tail vein injection on day 1 and again on day 6 post-bone marrow transplant. Mice are sacrificed at day 21 and skin and other tissues are collected, measured for thickening, and analyzed for collagen and immune cells.

### Example 9

### Efficacy of Agents that Decrease the Effective Concentration of Bioactive Lipids in Animal Allograft Models

Cardiac Allografts:

To determine the therapeutic effects of anti S1P antibody and other agents that decrease the effective concentration of bioactive lipids in preventing allograft rejection, these compounds are tested for activity in a murine vascularized heterotopic heart transplant model. Hearts from Balb/c mice are transplanted into the abdominal cavity of C3H mice as primary vascularized grafts essentially as described by Isobe et al., Circulation 1991, 84, 1246-1255. Test compounds are administered by injection into tail vein, or by continuous pump and allograft survival time is monitored by detection of a second heartbeat. Mean survival time of the allograft is expected to increase with anti-S1P antibody or other agents that decrease the effective concentration of bioactive lipids.

Renal Allografts:

A well-established model to study chronic rejection in renal allografts is the F344 to LEW rat model. All LEW recipients of F344 grafts develop acute rejection at approximately day 30 resulting in 50% graft loss. The surviving animals show histopathological and functional characteristics of CR from day 50. Joosten, S. A. et al., (2002) American Journal of Pathology 160:1301-1310. To determine the therapeutic effects of anti S1P monoclonal antibody and other agents that decrease the effective concentration of bioactive lipids in preventing allograft rejection, these compounds are tested for activity in the F344 to LEW rat model, essentially as described by Joosten et al. (supra).

Corneal Allografts:

Corneal transplantation (penetrating keratoplasty (PK)) is the most successful tissue transplantation procedure in humans, yet corneal allograft rejection is still the leading cause of corneal graft failure. [Ing J J et al. (1998), Ophthalmology, vol 105: 1855-1865]. Recently it has been discovered that CD4(+) T cells function as directly as effector cells and not helper cells in the rejection of corneal allografts. [Hegde S et al. (2005), Transplantation, vol 79: 23-31]. Murine studies have shown increased numbers of neutrophils, macrophage and mast cells in the stroma of corneas undergoing rejection. Yamagami S et al. (2005), Mol Vis, vol 11, 632-40.

FTY720 is an immunosuppressive drug that acts by altering lymphocyte trafficking; its immune-modulating effects are mediated by binding to some of the S1P receptors expressed on lymphocytes. [Bohler T et al. (2005), Transplantation, vol 79: 492-5]. FTY treated mice showed a significant prolongation of orthotopic corneal-graft survival when administered orally. [Zhang et al. (2003), Transplantation, 76: 1511-3]. FTY oral treatment also significantly delayed rejection and decreased its severity in a rat-to-mouse model of corneal xenotransplantation [Sedlakova et al. (2005), Transplantation, 79: 297-303]. Given the known pathogenesis of allograft rejection combined with the data suggesting that modulating the effects of the S1P signaling can improve corneal graft survival, it is believed that agents, for example, anti-S1P monoclonal antibody or other antibodies, that decrease the effective concentration of bioactive lipids, will also be useful in treatment of immunologic conditions such as allograft rejection, for example by attenuating the immune response, and thus will likely improve corneal graft survival. These agents are administered by injection into the tail vein or administered directly into the eye and are expected to prolong graft survival.

### Example 10

### Efficacy of Agents that Decrease the Effective Concentration of Bioactive Lipids in Animal Models of Glomerulonephritis

Immune diseases of the glomerulus, such as glomerulonephritis, are among the major causes of end-stage renal disease. These diseases share a progressive course characterized by fibrosis and inflammation of the tubulointerstitial compartment. For discussion see Peters et al., (2004), Kidney Intl. 66: 1434-1443. It is believed that agents, such as anti-S1P antibody or other agents that decrease the effective concentration of bioactive signaling lipids, will be particularly effective in conditions characterized by both a scarring and an autoimmune and/or inflammatory component.

In a rat model of glomerulonephritis, FTY720 treatment reduced circulating lymphocyte counts as well as renal lymphocyte infiltration. The course of disease progression was slowed significantly. Peters et al., supra. Because FTY is an S1P receptor antagonist, and therefore blocks S1P signaling, it is believed that agents that bind bioactive signaling lipids, such as lysolipids S1P and LPA, and reduce their effective concentration, will also demonstrate efficacy in treatment of glomerulonephritis, other immune-based kidney diseases and other conditions characterized, at least in part, by an aberrant, excessive or unwanted immune response.

Mouse models for glomerulosclerosis, a model system for glomerulonephritis, exist. Gao et al. (2004) Molec. Cell. Biol. 24: 9899. The effect of anti S1P monoclonal antibody on renal fibrosis and inflammation is tested in a mouse model of glomerulosclerosis essentially according to Gao. Because of its effect on both the immune response and fibrosis, anti-S1P monoclonal antibody and other agents that decrease the effective concentration of bioactive lipids are expected to be particularly effective at slowing renal autoimmune disease progression.

### Example 11

### Formulation Stability Study

### 1 Introduction

This example describes experiments to assess the stability of several formulations containing the humanized monoclonal antibody LT1009, which is reactive against the bioactive signaling lipid sphingosine 1-phosphate (S1P). LT1009 is an engineered full-length IgG1k isotype antibody that contains two identical light chains and two identical heavy chains, and has a total molecular weight of about 150 kDa. The complementarity determining regions (CDRs) of the light and heavy chains were derived from a murine monoclonal antibody generated against S1P, and further include a Cys to Ala substitution in one of the CDRs. In LT1009, human framework regions contribute approximately 95% of the total amino acid sequences in the antibody, which binds S1P with high affinity and specificity.

The purpose of the testing described in this example was to develop one or more preferred formulations suitable for systemic administration that are capable of maintaining stability and bioactivity of LT1009 over time. As is known, maintenance of molecular conformation, and hence stability, is dependent at least in part on the molecular environment of the protein and on storage conditions. Preferred formulations should not only stabilize the antibody, but also be tolerated by patients when injected. Accordingly, in this study the various formulations tested included either 11 mg/mL or 42 mg/mL of LT1009, as well as different pH, salt, and nonionic surfactant concentrations. Additionally, three different storage temperatures (5° C., 25° C., and 40° C.) were also examined (representing actual, accelerated, and temperature stress conditions, respectively). Stability was assessed using representative samples taken from the various formulations at five different time points: at study initiation and after two weeks, 1 month, 2 months, and 3 months. At each time point, testing involved visual inspection, syringeability (by pulling through a 30-gauge needle), and size exclusion high performance liquid chromatography (SE-HPLC). Circular dichroism (CD) spectroscopy was also used to assess protein stability since above a certain temperature, proteins undergo denaturation, followed by some degree of aggregate formation. The observed transition is referred to as an apparent denaturation or “melting” temperature (Tₘ) and indicate the relative stability of a protein.

### 2. Materials and Methods

a. LT1009

The formulation samples (˜0.6 mL each) were generated from an aqueous stock solution containing 42 mg/mL LT1009 in 24 mM sodium phosphate, 148 mM NaCl, pH 6.5. Samples containing 11 mg/mL LT1009 were prepared by diluting a volume of aqueous stock solution to the desired concentration using a 24 mM sodium phosphate, 148 mM NaCl, pH 6.5, solution. To prepare samples having the different pH values, the pH of each concentration of LT1009 (11 mg/mL and 42 mg/mL) was adjusted to 6.0 or 7.0 with 0.1 M HCl or 0.1 M NaOH, respectively, from the original 6.5 value. To prepare samples having different NaCl concentrations, 5 M NaCl was added to the samples to bring the salt concentration to either 300 mM or 450 mM from the original 148 mM. To prepare samples having different concentrations of nonionic surfactant, polysorbate-80 was added to the samples to a final concentration of either 200 ppm or 500 ppm. All samples were aseptically filtered through 0.22 μm PVDF membrane syringe filters into sterile, depyrogenated 10 mL serum vials. The vials were each then sealed with a non-shedding PTFE-lined stopper that was secured in place and protected from contamination with a crimped on cap. Prior to placement into stability chambers, the vials were briefly stored at 2-8° C.; thereafter, they were placed upright in a stability chamber adjusted to one of three specified storage conditions: 40° C. (±2° C.)/75% (±5%) relative humidity (RH); 25° C. (±2° C.)/60% (±5%) RH; or 5° C. (±3° C.)/ambient RH. A summary of the formulation variables tested appears in Table 3, below.

TABLE 3
Formulation Summary

| LT1009, 11 mg/mL | | | LT1009, 42 mg/mL | | |
|---|---|---|---|---|---|
| Polysorbate 80 | NaCl | pH | Polysorbate 80 | NaCl | pH |
| 0.02% | 148 mM | 7 | 0.02% | 148 mM | 7 |
| Polysorbate | NaCl | 6.5 | Polysorbate | NaCl | 6.5 |
| | | 6 | | | 6 |
| | 300 mM | 7 | | 300 mM | 7 |
| | NaCl | 6.5 | | NaCl | 6.5 |
| | | 6 | | | 6 |
| | 450 mM | 7 | | 450 mM | 7 |
| | NaCl | 6.5 | | NaCl | 6.5 |
| | | 6 | | | 6 |
| 0.05% | 148 mM | 7 | 0.05% | 148 mM | 7 |
| Polysorbate | NaCl | 6.5 | Polysorbate | NaCl | 6.5 |
| | | 6 | | | 6 |
| | 300 mM | 7 | | 300 mM | 7 |
| | NaCl | 6.5 | | NaCl | 6.5 |
| | | 6 | | | 6 |
| | 450 mM | 7 | | 450 mM | 7 |
| | NaCl | 6.5 | | NaCl | 6.5 |
| | | 6 | | | 6 |

b. Taking of Samples

Samples of each formulation were analyzed according to the schedule listed in Table 4, below. One vial was used for each storage condition for all time points. On a date when samples were to be taken, vials were pulled from each stability chamber and 150 μL of each sample were transferred into correspondingly labeled separate vials that were placed on the bench for 1 hour prior to testing. The original vial was immediately placed back into the specified stability chamber after withdrawing the aliquot to be tested.

TABLE 4
Drug Product Formulation Study Stability Matrix

| Storage | Intervals (months) | | | | |
|---|---|---|---|---|---|
| Conditions | T = 0 | 0.5 | 1 | 2 | 3 |

Protein Concentration LT1009, 11 mg/mL

| | | | | | |
|---|---|---|---|---|---|
| 40° C. | x, y | x, y | x | x | x, y |
| 25° C. | | x, y | x | x | x, y |
| 5° C. | | x, y | x | x | x, y |

Protein Concentration LT1009, 42 mg/mL

| | | | | | |
|---|---|---|---|---|---|
| 40° C. | x, y | x, y | x | x | x, y |
| 25° C. | | x, y | x | x | x, y |
| 5° C. | | x, y | x | x | x, y |

x = Appearance, pH, SDS-PAGE, SE-HPLC, UV OD-280, IEF
y = Syringeability (performed by aseptically drawing 200 μL of a sample with a 30-gauge needle connected to a disposable 1-mL syringe)

c. Analytical Procedures

For a given time point, aliquots from each sample were subjected to a series of standard analyses, including visual inspection, syringeability, pH, SDS-PAGE (under both reducing and non-reducing conditions), SE-HPLC, and IEF. Protein concentrations were determined by UV spectroscopy (OD-280). Circular dichroism (CD) studies were also performed.

Circular Dichroism spectroscopy was performed separately from the formulation studies. An Aviv 202 CD spectrophotometer was used to perform these analyses. Near UV CD spectra were collected from 400 nm to 250 nm. In this region, the disulfides and aromatic side chains contribute to the CD signals. In the far UV wavelength region (250-190 nm), the spectra are dominated by the peptide backbone. Thermal denaturation curves were generated by monitoring at 205 nm, a wavelength commonly used for b-sheet proteins. Data was collected using 0.1 mg/ml samples with heating from 25° C. to 85° C. Data were collected in 1° C. increments. The total time for such a denaturation scan was between 70 and 90 minutes. The averaging time was 2 seconds.

### 3. Results and Discussion

For all samples analyzed, visual appearance did not change over time. Likewise, syringeability testing demonstrated that samples could be pulled into a syringe equipped with a 30-gauge needle without difficulty. The results of the various analytical tests were consistent, and SE-HPLC was determined to be an excellent stability-indicating method for LT1009. These results showed that increasing salt concentration reduced both the generation of aggregates and the generation of smaller non-aggregate impurities. It was also found that decreasing pH also reduced aggregate and impurity formation. In addition, it was determined that increasing the polysorbate-80 concentration above 200 ppm did not further stabilize LT1009. Figure X illustrates the results of the SE-HPLC experiments performed on samples containing 11 mg/mL LT1009. Comparable results were obtained for samples containing 42 mg/mL LT1009, although lower LT1009 concentrations showed less potential for aggregate formation as compared to the higher concentration, indicating that the antibody appeared to be slightly less stable under all conditions tested at the higher concentration.

From the circular dichroism studies, it was found that LT1009 adopts a well-defined tertiary structure in aqueous solution, with well-ordered environments around both Tyr and Trp residues. It also appeared that at least some of the disulfides in antibody molecules experience some degree of bond strain, although this is not uncommon when both intra- and inter-chain disulfides are present. The secondary structure of LT1009 was found to be unremarkable, and exhibited a far UV CD spectrum consistent with β-sheet structure. The observed transition is referred to as an apparent denaturation or “melting” temperature (Tₘ). Upon heating, LT1009 displayed an apparent Tₘ of approximately 73° C. at pH 7.2. The apparent Tₘ increased to about 77° C. at pH 6.0. These results indicate that a slightly acidic pH could enhance long-term stability of aqueous formulations of LT1009. Addition of NaCl and/or polysorbate-80 also provided additional stabilization.

Together, the data from these experiments indicate that LT1009 is most stable around pH 6 and 450 mM NaCl independent of antibody concentration. Indeed, SE-HPLC testing indicated that increasing the salt concentration to 450 mM and decreasing the pH to 6.0 while maintaining the polysorbate-80 concentration at 200 ppm had a very beneficial effect on the stability of LT1009. Inclusion of polysorbate-80 above 200 ppm had no further mitigating effect against aggregate formation, probably because it was already above its critical micelle concentration at 200 ppm. While not wishing to be bound by any particular theory, the fact that aggregate formation in LT1009 was reduced with increasing salt concentration under the studied conditions could indicate that aggregate formation is at least in part based more on ionic interactions between molecules rather than hydrophobic interactions. The observation that lowering the pH from 7 to 6 also reduces aggregate formation could be explained by reduced hydrophobicity of the amino acid histidine at the lower pH. Finally, the observed increased tendency of aggregate formation at increased LT11009 concentration can simply be explained by the greater chance of molecules hitting each other at the right time at the right place for aggregate formation.

In view of these experiments, a preferred aqueous LT1009 formulation is one having 24 mM phosphate, 450 mM NaCl, 200 ppm polysorbate-80, pH 6.1. The relatively high tonicity of this formulation should not pose a problem for systemic applications since the drug product will likely be diluted by injection into iv-bags containing a larger volume of PBS prior to administration to a patient.

The disclosures of each and every patent, patent application, and publication cited herein are hereby incorporated herein by reference in their entirely.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of decreasing an aberrant, excessive or undesired immune response in an animal comprising administering to said animal an agent that binds a bioactive lipid and reduces the effective concentration of said bioactive lipid.

2. The method of claim 1 wherein the bioactive lipid is a sphingolipid, a sphingolipid metabolite, a lysolipid or a lysolipid metabolite, optionally wherein the lysolipid is S1P, LPA or a variant of S1P or LPA.

3. The method of claim 1 wherein the agent is an antibody, antibody fragment, or antibody variant; a receptor fragment, ion channel fragment, aptamer or the domain of an enzyme that binds a bioactive lipid, wherein the antibody is optionally a monoclonal antibody, optionally a humanized monoclonal antibody.

4. The method of claim 1 wherein the aberrant, excessive or undesired immune response is an autoimmune response.

5. A method of treating a disease or condition in an animal, said disease or condition being characterized by an aberrant, excessive or undesired immune response, comprising administering to said animal an agent that binds a bioactive lipid and reduces the effective concentration of said bioactive lipid.

6. The method of claim 5 wherein the bioactive lipid is a sphingolipid, a sphingolipid metabolite, a lysolipid or a lysolipid metabolite, and optionally wherein the lysolipid is S1P, LPA or a variant of S1P or LPA.

7. The method of claim 5 wherein the agent is an antibody, an antibody fragment or an antibody variant.

8. The method of claim 7 wherein the antibody is a monoclonal antibody, optionally a humanized monoclonal antibody.

9. The method of claim 5 wherein the aberrant, excessive or undesired immune response is an autoimmune response.

10. The method of claim 5 wherein the disease or condition is characterized by paralysis or ataxia, and wherein said paralysis or ataxia is decreased by said treatment.

11. A method of decreasing paralysis, ataxia, demyelination or neurodegeneration in an animal, wherein said paralysis, ataxia, demyelination or neurodegeneration results from an autoimmune disease or condition, comprising administering to said animal an agent that binds a bioactive lipid and reduces the effective concentration of said bioactive lipid, so that said paralysis or ataxia is decreased.

12. The method of claim 11 wherein the bioactive lipid is a sphingolipid, a sphingolipid metabolite, a lysolipid or a lysolipid metabolite, and optionally wherein the lysolipid is S1P, LPA or a variant of S1P or LPA.

13. The method of claim 11 wherein the agent is an antibody, an antibody fragment or an antibody variant.

14. The method of claim 13 wherein the antibody is a monoclonal antibody, optionally a humanized monoclonal antibody.

15. The method of any one of claims 1-14 wherein said animal is a human.

16. The method of claim 15 wherein said human has, or is believed to have, multiple sclerosis.

17. The method of any one of claims 1-16 wherein the agent that binds a bioactive lipid is administered in combination with a therapeutic agent that is administered for treatment of multiple sclerosis or a symptom thereof.

18. The method of claim 17 wherein the therapeutic agent is a disease modifying agent for treatment of multiple sclerosis, a corticosteroid or a therapeutic agent that is administered for treatment of a primary or secondary symptom of multiple sclerosis.

19. The method of claim 18 wherein the disease modifying agent for treatment of multiple sclerosis is an immunomodulator or an immunosuppressant, wherein the immunomodulator may be beta interferon 1b, beta interferon 1a, glatiramer acetate or natalizumab, and the immunosuppressant may be mitoxantrone or FTY720.
